(19) United States
(12) Patent Application Publication
(10) Pub. No.: US 2025/0352524 A1
(43) Pub. Date: Nov. 20, 2025

(54) PREVENTION OF METASTATIC OUTGROWTH USING TEAD INHIBITORS

(71) Applicant: Georgetown University, Washington, DC (US)

(72) Inventors: Ghada M. Sharif, Arlington, VA (US); Anna T. Riegel, Washington, DC (US); Anton Wellstein, Washington, DC (US)

(73) Assignee: Georgetown University, Washington, DC (US)

(21) Appl. No.: 19/208,262

(22) Filed: May 14, 2025

Related U.S. Application Data
(60) Provisional application No. 63/647,126, filed on May 14, 2024.

(51) Int. Cl. A61K 31/44; A61K 9/00; A61P 35/04

(52) U.S. Cl. CPC A61K 31/44; A61K 9/0053; A61P 35/04

(57) ABSTRACT

Method of treating secondary cancer in a lung of a subject in need thereof, comprising administering a pharmaceutical composition comprising an effective amount of an inhibitor of transcriptional enhanced associate domain (TEAD) to the subject. In addition, methods of inhibiting growth of a secondary tumor in a lung of a subject in need thereof, of increasing number of T-cells at a secondary tumor in a lung of a subject in need thereof, of increasing number of alveolar macrophages in a lung of a subject in need thereof, of decreasing number of infiltrating monocytes/macrophages in a lung of a subject in need thereof, methods of reducing lung metastases in a subject in need thereof, method of inducing polarization of one or more M2 macrophages to M1 macrophages in the lung of a subject with lung cancer, and methods of activating IL12 signaling in lung of a subject with lung cancer.

### CROSS-REFERENCE TO RELATED APPLICATIONS

This application claims the benefit of U.S. Provisional Application No. 63/647,126, filed on May 14, 2024, which is incorporated herein by reference in its entirety.

### STATEMENT REGARDING FEDERALLY SPONSORED RESEARCH OR DEVELOPMENT

This invention was made with government support under CA051008 awarded by the National Institutes of Health. The government has certain rights in the invention.

### SEQUENCE LISTING

The instant application contains a Sequence Listing, which has been submitted electronically in XML format and is hereby incorporated by reference in its entirety. Said XML copy, created on May 13, 2025, is named Georgtown_047_US1-Sequence_Listing, and is 13,195 bytes in size.

### BACKGROUND

The lung is the second most frequent site of distant metastasis. It is estimated that 20% to 54% of malignant tumors developing elsewhere in the body would have pulmonary metastasis (Zhao et al., 2017; Stella et al., 2019). Among cancers that can metastasize to the lungs are colorectal, head and neck, urologic (kidney, ureter, prostates, testes), gastrointestinal non-colorectal, and breast (Caparica et al., 2016). The spread of tumors to the lung typically occurs by a hematogenous route, which is often seen in tumors with venous drainage into lungs; by a lymphatic route, either antegrade lymphatic invasion through the diaphragm and/or pleural surfaces or retrograde lymphatic spread from hilar nodal metastases; or by direct invasion to the pleura (Stella et al., 2019).

The prognosis of lung metastasis varies depending on the type of tumor. Distant metastasis to the lung generally categories a tumor as Stage IV.

Among the cancers associated with a more deadly prognoses for lung metastasis is triple negative breast cancer (TNBC), which is a subtype of breast cancer characterized by no expression of estrogen receptor (ER), progesterone receptor (PR), and human epidermal growth factor receptor-2 (HER2). TNBC is an aggressive disease with short overall survival rate (Kesireddy et al., 2024). Patients with metastatic TNBC are treated with a combination of chemotherapy, immunotherapy, as well as select targeted therapies, which only benefit a small subset of patients (Li et al., 2019).

TNBC exhibits a greater propensity to metastasize compared to other subtypes, with approximately 50% of patients with TNBC developing distant metastasis (Yin et al., 2020). The lungs are among the most common sites of distant metastasis associated with TNBC, accounting for 40% of the cases of metastasis (Wang et al., 2022). Development of lung metastasis often occurs within five years of the initial breast cancer diagnosis and leads to significant morbidity and mortality of patients with lung metastases, 60% to 70% will succumb to their disease with a median survival of about 25 months (Rashad & Takabe, 2012; Xiao et al., 2018).

Thus, a therapy that can prevent or inhibit distant metastasis to the lung can be critical to enhancing cancer survival, especially in patients with TNBC.

### SUMMARY OF THE INVENTION

The present invention is based, in part, on a series of important discoveries that are described in more detail in the Examples section of this patent specification. For example, a population of cancer cells (i.e., cells expressing AIB1A4, an N-terminally truncated isoform of the oncogene Amplified In Breast Cancer 1), which enable the successful seeding and colonization of other cancer cells in the lungs, was found to display an enrichment in transcriptional enhanced associate domain (TEAD) binding motifs. Use of a TEAD inhibitor reduced the invasive ability of tumor spheres in vitro, and suppressed the development of lung metastases in vivo. It was also found that the TEAD inhibitor caused a shift in lung resident macrophages and T-cell infiltration and activation in treated animals. Further, TEAD inhibition suppressed pro-tumor inflammation and M2-like macrophages, and increased interleukin-12 (IL12) signaling in the lung tissue and production from tissue resident macrophages, which enhanced macrophage-T-cell crosstalk and increased Th1 CD4+ frequencies and infiltration of CD8+ T-cells into the lung. Building on these discoveries, and other discoveries presented herein, the present invention provides a variety of new and improved methods and uses for TEAD inhibitors to inhibit metastatic outgrowth into lungs.

Accordingly, in one aspect, the present invention provides a method of treating secondary cancer in the lung of a subject in need thereof, comprising administering a pharmaceutical composition comprising an effective amount of TEAD inhibitor to the subject. In another aspect, the present invention provides a pharmaceutical composition comprising an effective amount of a TEAD inhibitor for use in treating secondary cancer in a lung of a subject in need thereof.

In one aspect, the present invention provides a method of inhibiting growth of a secondary tumor in a lung of a subject in need thereof, the method comprising administering a pharmaceutical composition comprising an effective amount of a TEAD inhibitor to the subject. In yet another the present invention provides a pharmaceutical composition comprising an effective amount of a TEAD inhibitor for use in inhibiting growth of a secondary tumor in a lung of a subject in need thereof.

In a further aspect, the present invention provides a method of increasing number of T-cells at a secondary tumor in a lung of a subject in need thereof, the method comprising administering a pharmaceutical composition comprising an effective amount of a TEAD inhibitor to the subject. In another aspect, the present invention provides a pharmaceutical composition comprising an effective amount of a TEAD inhibitor for use in increasing number of T-cells at a secondary tumor in a lung of a subject in need thereof.

In some embodiments, the subject comprises a primary tumor in a tissue selected from breast, bone, esophagus, colon, rectum, kidney, cervix, prostate, larynx, liver, pancreas, brain, lung, and skin. In certain embodiments, the subject comprises a primary tumor in breast tissue. In particular embodiments, the subject has TNBC.

In one aspect, the present invention provides a method of increasing number of alveolar macrophages in the lung of a subject in need thereof, the method comprising administering a pharmaceutical composition comprising an effective amount of a TEAD inhibitor to the subject. In another aspect, the present invention provides a pharmaceutical composition comprising an effective amount of a TEAD inhibitor for use in increasing number of alveolar macrophages in a lung of a subject in need thereof.

In yet another aspect, the present invention provides a method of decreasing number of infiltrating monocytes/macrophages in a lung of a subject in need thereof, the method comprising administering a pharmaceutical composition comprising an effective amount of a TEAD inhibitor to the subject. In a further aspect, the present invention provides a pharmaceutical composition comprising an effective amount of a TEAD inhibitor for use in decreasing number of infiltrating monocytes/macrophages in a lung of a subject in need thereof.

In some embodiments, the subject is at risk of developing a secondary tumor in the lung.

In certain embodiments, the subject comprises a primary tumor in a tissue selected from breast, bone, esophagus, colon, rectum, kidney, cervix, prostate, larynx, liver, pancreas, brain, lung, and skin. In certain embodiments, the subject comprises a primary tumor in breast tissue. In particular embodiments, the subject has TNBC.

In one aspect, the present invention provides a method of reducing lung metastases in a subject in need thereof, the method comprising administering a pharmaceutical composition comprising an effective amount of an inhibitor of a TEAD inhibitor to the subject. In another aspect, the present invention provides a pharmaceutical composition comprising an effective amount of a TEAD inhibitor for use in reducing lung metastases in a subject in need thereof.

The subject may have metastatic cancer of the lung. In some embodiments, the subject has primary cancer selected from breast, bone, esophageal, colon, rectal, kidney, cervical, prostate, larynx, liver, pancreatic, brain, lung, and skin cancer. In certain embodiments, the primary cancer is breast cancer, for example, TNBC.

In a further aspect, the present invention provides a method of inducing T-cell activation in the lung of a subject with lung cancer, the method comprising administering a pharmaceutical composition comprising an effective amount of a TEAD inhibitor to the subject. In another aspect, the present invention provides a pharmaceutical composition comprising an effective amount of a TEAD inhibitor for use in inducing T-cell activation in the lung of a subject with lung cancer.

In an additional aspect, the present invention provides a method of inducing polarization of one or more M2 macrophages to M1 macrophages in the lung of a subject with lung cancer, the method comprising administering a pharmaceutical composition comprising an effective amount of a TEAD inhibitor to the subject. In another aspect, the present invention provides a pharmaceutical composition comprising an effective amount of a TEAD inhibitor for use in inducing polarization of one or more M2 macrophages to M1 macrophages in the lung of a subject with lung cancer.

The lung cancer may be a primary cancer or a metastatic cancer. In embodiments in which the lung cancer is a metastatic cancer, the subject may have a primary cancer selected from breast, bone, esophageal, colon, rectal, kidney, cervical, prostate, larynx, liver, pancreatic, brain, lung, and skin cancer. In certain embodiments, the primary cancer is breast cancer, for example, TNBC.

In embodiments of the invention, the pharmaceutical composition is administered systemically. In certain embodiments, the pharmaceutical composition is administered via a route selected from intravenously, orally, intraperitoneally, intramuscularly, intradermally, intrathecally, subcutaneously, and nasally.

### BRIEF DESCRIPTION OF THE FIGURES

FIG. 1 shows results from injecting a mixture of parental ductal carcinoma in situ (DCIS) with a small population of AIB1Δ4-expressing DCIS cells (“DCIS-Δ4”) that have high TEAD activity into the mammary fat pad of mice, and administering either TEAD inhibitor VT107 (iTEAD) or a vehicle, as described in Example 1. Panel A compares primary tumor size between mice administered iTEAD and mice administered the vehicle. Panel B presents representative images of metastases in the lung and compares the number of iTEAD-administered mice and vehicle-adminstered mice with lung metastases (** indicates p<0.01). Panel C presents representative images of metastases and cancer cells in the lungs of iTEAD-administered mice and vehicle-adminstered mice. These results show that TEAD inhibition had no effect on primary tumors (Panel A), yet significantly reduced metastasis in the lungs (Panel B). Further, seeded cells were unable to outgrow in the iTEAD-administered lungs (Panel C).

FIG. 2 shows results of Gene Set Enrichment Analysis (GSEA) of altered signaling pathways upon iTEAD treatment in vivo, as described in Example 1. A negative enrichment score indicates downregulation. The p-value cutoff is less than 0.05. The resuls show a downregulation of Toll Like Receptor 4 signaling in iTEAD-administered mice.

FIG. 3 shows results from injecting E0771 cells into the mammary fat pad of C57/BL6 mice, and administering either iTEAD or a vehicle, as described in Example 2. Panel A compares primary tumor size between mice administered iTEAD and mice administered the vehicle. Panel B compares the number of iTead-administered mice and vehicle-adminstered mice with lung metastases. Panel C compares presence of alveolar macrophages and infiltrating macrophages measured by flow cytometry in the lung tissue of iTEAD-administered mice and vehicle-adminstered mice (* indicates p<0.05). Panel D compares presence of CD8+ T-cells measured by flow cytometry in the lung tissue of iTead-administered mice and vehicle-adminstered mice. These results show that there was no change on E0771 primary tumor size with iTEAD treatment (Panel A), yet the number of mice with lung metastasis was reduced (Panel B). In addition, there was a significant increase in alevolar macrophages a significant decrease in infiltrating macrophages (Panel C), and a higher numbers of cytotoxic T-cells (Panel D), in iTEAD-administered mice as compared to vehicle-administered mice.

FIG. 4 shows results of immunohistochemistry of CD8 on lung fixed tissue from tumor-bearing mice (from FIG. 3), as described in Example 2. Panel A compares presence of CD8+ T-cells in the lung tissue of iTEAD-administered mice and vehicle-adminstered mice (*** indicates p<0.001). Panel B presents representative images of lung metastases from iTEAD-administered mice and vehicle-adminstered mice. These results show that the number of CD8+ T-cells in the lungs was significantly greater in iTEAD-administered mice as compared to vehicle-adminstered mice (Panel A), and that CD8+ T-cells infiltrated into lung metastases of the iTEAD-administered mice (Panel B).

FIG. 5 shows results from administering iTEAD or a vehicle to naíve C57/BL6 mice for two weeks, as described in Example 3. The figure compares presence of alveolar macrophages and infiltrating monocytes/macrophages measured by flow cytometry in the lung and spleen tissue, and in blood. The results show a shift in lung alveolar versus infiltrating monocytes/macrophages in the iTEAD-administered mice, and no change in the spleen or blood (* indicates p<0.05; ** indicates p<0.01).

FIG. 6 shows the effect of iTEAD or a TLR4 inhibitor (iTLR4) on DCIS cells- and DCIS-Δ4 cells-mixed 3D spheres embedded in a collagen:matrigel mix, as described in Example 3. Panel A shows representative images of a spheroid of DCIS cells (red cells) and DCIS-Δ4 cells (green cells), and compares the number of invasive cells per spheroid in iTEAD and control groups (* indicates p<0.05). Panel B shows representative images of a spheroid of DCIS cells (red cells) and DCIS-Δ4 cells (green cells), and compares invasion area of cells per spheroid in iTLR4 and control groups (**** indicates p<0.0001). The results show that iTEAD (Panel A) and iTLR4 (Panel B) reduced collective invasion of the DCIS and DCIS-Δ4 mixed 3D spheres.

FIG. 7 shows a diagram depicting T-cell/macrophage mixing with tumor spheres to monitor T-cell mediated cancer cell killing, as described in Example 4. The figure presents images of 4T1 cells spheres mixed with either T-cells alone or T-cells and macrophages with or without administration of iTEAD. H₂O₂-administration was used as a positive control for 4T1 sphere killing.

FIG. 8 demonstrates effect of TEAD inhibition in mice injected with mammary carcinoma cell line E0771, as described in Example 6. Panel A shows Real-Time Cellular Analysis (RTCA) of E0771 cells measured by electric impedance sensing of attached cells (cell index) over time with increasing doses of VT107 (Mean±SD, student t-test). Panel B shows body weight of C57/B16 mice bearing E0771 tumors with VT107 or vehicle treatment. Panel C shows rate of tumor recurrence between treatment groups 2 weeks after E0771 tumor resection at week 4 (Student t-test). Panels D and E show volcano plots depicting change in tumor gene expression (Panel D) and lung gene expression (Panel E) of VT107-treated versus the control group in tumor bearing mice. Panel F shows IPA depicting differences in signaling pathways in the tumor naïve lungs of VT107-treated versus vehicle treated mice that have a p-val <0.05 and a |z-score|>2. Panel G shows volcano plots depicting changes in lung and spleen gene expression between VT107 or vehicle treatment in tumor naïve mice. Circles in the bracketed sections of the volcano plots indicate regulated genes with −Log p-value >1.3 and |log 2FC|=2.

FIG. 9 demonstrates effect of TEAD inhibition on lung immune microenvironment and IL12 signaling, as described in Example 6. Panel A shows schematic of mammary fat pad (MFP) injection of E0771 cells in C57/B16 mice. Oral gavage with 30 mg/kg of the iTEAD VT107 or vehicle started when tumors were palpable (3 weeks). Panel B shows primary tumor volumes measured after surgical resection at 4 weeks (Mean±SD, student t-test, n=4-5 mice per group). Panel C shows quantification of the number of C57/B16 mice that developed lung metastases determined by the histopathological analysis of lung tissue sections stained with hematoxylin and eosin (H&E) in the VT107-treated versus control group. Representative images of lung sections showing presence or absence of metastases (Fisher's exact test; scale bar is 100 μm). Panel D shows ingenuity pathway analysis (IPA) showing altered signaling pathways in the lungs of VT107-treated versus vehicle treated mice that have a p<0.05 and a |z-score|>1.3. The z-score indicate the directionality of the altered pathway, positive z-score value indicates upregulation while a negative value indicates downregulation of a signaling pathway. Panel E shows schematic of BALB/c mice that were injected intravenously (IV) with 4T1 cells and treated with either vehicle or 30 mg/kg VT107 daily by oral gavage. Panel F shows quantification of the number of BALB/c mice that developed lung metastases, determined by the histopathological analysis of H&E lung sections in the VT107 treated versus control group. Representative H&E-stained lung sections showing metastases (Fisher's exact test). Panel G shows experimental scheme of tumor naïve C57/B16 mice that were treated with 30 mg/kg of the iTEAD VT107 or vehicle for two weeks before tissue collection (n=5 mice per group). Panel H shows IPA depicting differences in signaling pathways in the tumor naïve lungs of VT107-treated versus vehicle treated mice that have a p-val <0.05 and a |z-score|>2. Panel I shows CIBERSORTx (Chen et al., 2018) analysis of mRNA from lungs of VT107-treated and vehicle-treated groups, indicating changes in the abundance of tissue cell types.

FIG. 10 demonstrates effect of TEAD inhibition on lung resident macrophages and IL12 production, as described in Example 6. Panel A shows flow cytometry analysis of alveolar macrophages and monocytes in the lung, spleen and blood of tumor naïve mice treated with VT107 or vehicle (Mean±SEM, student t-test). Panel B shows blot of cytokine array and quantified data depicting changes in secreted factors in the conditioned media of isolated lung macrophages (MF_Lungs) or bone marrow-derived macrophages (MF_BM) treated with vehicle or 1 μM VT107 for 24 hours (Mean+SEM, student t-test). Panel C shows flow cytometry analysis depicting percent of M1-like (CD86+) vs M2-like (CD206+) cells relative to the total macrophage population in the lungs of VT107 and vehicle-treated tumor bearing mice from FIG. 9, Panel A (Mean±SEM, student t-test). Panel D shows flow cytometry analysis depicting percent of M1-like (CD86+) vs M2-like (CD206+) cells relative to the total alveolar macrophage population in the lungs of VT107 and vehicle-treated tumor bearing mice (Mean±SEM, student t-test). Panel E shows immunohistochemistry (IHC) quantification for CD8 T-cells in lung sections from vehicle vs iTEAD-treated tumor bearing mice (Mean±SEM, Mann Whitney t-test, n=10 fields/lung). Representative images show differential infiltration of CD8+ T-cells into lung metastases between treatment groups. Scale bar is 50 μm. Panel F shows IHC quantification of T-bet+ cells in the lungs of vehicle vs iTEAD-treated mice from FIG. 9, Panel E at week 2 (Mean±SEM, Mann Whitney t-test, n=10 fields/lung, n=5 mice per group). Representative images show the absence or presence of T-bet+ cells in lungs of vehicle or VT107 treated mice respectively. Scale bar is 20 μm.

FIG. 11 demonstrates effect of TEAD inhibition on immune cells, as described in Example 6. Panel A shows flow cytometry analysis depicting change in the total macrophage, neutrophil, eosinophils, or dendritic cell populations in tumor naïve mice treated with VT107 or vehicle. Panel B shows flow cytometry analysis depicting change in lymphoid cells of tumor naïve mice treated with VT107 or vehicle. Panel C shows flow cytometry analysis of the alveolar macrophage and monocyte population in the lung and spleen of tumor bearing mice that were treated with VT107 versus vehicle from FIG. 9, Panel A (Student t-test). Panel D shows flow cytometry analysis of the alveolar macrophage and monocyte population in the lung and spleen of tumor bearing mice that were treated with VT107 versus vehicle for two weeks from FIG. 9, Panel E (Student t-test). Panel E shows quantification of IHC of T-cell marker CD8 in the tumors of VT107-treated versus the control group (n=5 fields/lung, student t-test). Panel F shows flow cytometry analysis of the lymphoid cells in the lung of balb/c mice that were treated with VT107 versus vehicle for two weeks from FIG. 1E. (Student t-test). Panel G shows flow cytometry analysis of CD4+ T-cell subtypes in the lung of balb/c mice that were treated with VT107 versus vehicle for two weeks from FIG. 9, Panel E (Student t-test).

FIG. 12 demonstrates effect of TEAD knockdown on macrophages crosstalk with cancer cells and metastatic seeds' outgrowth in the lung of mice characterized by their non-obese diabetic background and severe combined immunodeficiency (NOD/SCID mice), as described in Example 6. Panel A shows changes in gene expression measured by qPCR in THP1 macrophages that were treated with 1 μM VT107 or vehicle. The gene expression of three TEAD target genes is downregulated confirming TEAD-related transcription inhibition by VT107. Panel B shows quantification of THP1 macrophages invasion into a matrix with 4T1 spheres. n=8-12 spheres per condition (Mean±SD, student t-test). THP1 macrophages harboring two different shRNAs for TEAD or control were stained with a green cell tracer. Panel C shows volcano plots depicting changes in gene expression in THP1 macrophages between shTEAD and shControl. Circles in the bracketed sections of the volcano plots indicate regulated genes with −Log p-value >1.3 and |log 2FC|=2. Panel D shows IPA depicting altered signaling pathways in shTEAD versus shControl THP1 macrophages with p-val<0.05 and a |z-score|>2. Panel E shows IPA depicting upstream regulators altered in shTEAD versus shControl THP1 macrophages with a −Log p-value >1.3 and a |z-score|>2. Panel F shows quantification of the number of NOD/SCID mice with lung metastases determined by histological analysis of lung tissue sections stained with H&E in VT107 treated and control mice. Representative fields of lung sections show metastatic lesions (Fisher's exact test; scale bar is 500 μm). Panel G shows immunofluorescent staining of DCIS:DCISA4 cell mix (red) in overt metastases or single cells in the lungs of mice that were treated with vehicle or VT107, respectively. The lower inset shows a magnified view of the lung tissue where single cancer cells were detected in VT107-treated mice (scale bar is 100 μm and 20 μm in inset).

FIG. 13 demonstrates effect of TEAD inhibition on NOD/SCID mice bearing DCIS:DCISA4 tumors, as described in Example 6. Panel A shows Western blot of pan-TEAD protein levels in THP1 monocytes infected with either control shRNA or two distinct shTEAD lentiviruses. GAPDH protein levels were used as a loading control. Panel B shows experimental scheme of MFP injection of DCIS:DCISA4 cells in NOD/SCID mice. Oral gavage with 30 mg/kg of the iTEAD VT107 or DMSO vehicle started at the same time as MFP inoculation and terminated after primary tumor removal. Panel C shows body weight of NOD/SCID mice bearing DCIS:DCISA4 tumors in the different treatment groups. Panel D shows primary tumor volumes were measured after surgical resection at 4 weeks (Mean±SD; student t-test, n=5 mice per group). Panel E shows RTCA of proliferation of DCIS cells measured by electric impedance sensing of attached cells (cell index) over time with increasing doses of VT107 (Mean±SD, student t-test). Panel F shows qPCR of human actin (cancer cells) normalized to mouse actin (stroma) in the lung (metastasis site) and liver (control organ) tissue of mice that were treated with VT107 or vehicle. Panel G shows IPA depicting altered signaling pathways in the lungs of VT107-treated versus vehicle treated NOD/SCID mice that have a p-val <0.05 and a |z-score|>1.3.

FIG. 14 demonstrates effect of TEAD inhibition on early macrophage-T-cell crosstalk via IL12, as described in Example 6. Panel A shows schematic of macrophage and T-cell isolation and downstream crosstalk analysis. T-cells were isolated from naïve mice spleens. Macrophages were isolated from the peritoneal cavity by lavage (MF-PL) and pretreated for 24 hrs with 1 μM VT107. Panel B shows blot of cytokine array and quantified data depicting changes in secreted factors in the conditioned media of T-cells and macrophages that were pretreated with 1 μM VT107 and mixed for 5 hours (Mean+SEM, student t-test). Panel C shows representative images of immunohistochemistry of cCas-3 on 4T1 spheres embedded in matrix. T-cell and peritoneal macrophages were added to the spheres with the indicated treatments (vehicle, 1 μM VT107, VT3989, or 0.1 mg/ml IL12 antibody) (scale bar is 10 μm). Panel D shows quantification of the cCas-3 IHC signal 24 hours after adding the immune cells to 4T1 spheres embedded in matrix. n=3-10 spheres per condition (Mean±SD, student t-test).

FIG. 15 demonstrates effect of TEAD inhibition on peritoneal macrophages, as described in Example 6. Panel A shows volcano plot of the altered gene expression in peritoneal macrophages that were treated in vitro with vehicle or 1 μM VT107 for 72 hours. Circles in the bracketed sections indicate regulated genes with −Log p-value >1.3 and |log 2FC|=2. Panel B shows IPA depicting altered signaling pathways in peritoneal macrophages that were treated with vehicle or 1 μM VT107 for 24 hours. −Log p-value >1.3 and |log 2FC|=2. Panel C shows blot of cytokine array and quantified data depicting changes in secreted factors in the conditioned media of T-cells or macrophages treated overnight with 1 μM VT107. Panel D shows RTCA of 4T1 cancer cells measured by electric impedance sensing of attached cells (cell index) over time. Macrophages were pretreated for 24 hrs with 1 μM VT107 washed then added with the T-cells to the wells+/−IL12 antibody (0.1 mg/ml) (Mean±SD, student t-test). Panel E shows RTCA of 4T1 cancer cells measured by electric impedance sensing of attached cells (cell index) over time. T-cells treated with 1 μM VT107 were added simultaneously with the cancer cells to monitor cytotoxicity+/−IL12 antibody (0.1 mg/ml) (Mean±SD, student t-test).

FIG. 16 demonstrate effect of TEAD inhibition on CD4 and CD8 T-cells and on Jurkat cells, as described in Example 6. Panel A shows flow cytometry analysis of IFNγ levels in CD4 and CD8 T-cells that were treated with VT107 or vehicle for 72 hrs (Student t-test). Panel B shows RTCA of 4T1 cells measured by electric impedance sensing of attached cells (cell index) over time. Jurkat cells treated with vehicle or 1 μM VT107 for 24 hours then added to the cancer cells to monitor their cytotoxicity (Mean±SD, student t-test). Panel C shows Western blot of pan-TEAD protein levels in Jurkat cells infected with either control shRNA or two distinct shTEAD lentiviruses. GAPDH protein levels were used as a loading control.

FIG. 17 demonstrates effect of TEAD inhibition on T-cells activation and degranulation, as described in Example 6. Panel A shows flow cytometry analysis depicting change in CD107+ T-cells, a degranulation marker. T-cells isolated from the spleen were co-cultured with 4T1 cancer cells and treated with either vehicle or 1 μM VT107 for 72 hrs. Panel B shows quantification of 4T1 cell number in spheres after 72 hours co-culture with T-cells that were pretreated with vehicle or 1 μM VT107 for 24 hours. Treatment continued at time of co-culture (Mean±SD, student t-test). Representative H&E images are shown (scale bar is 10 μm). Panel C shows quantification of cleaved caspase 3 (cCas-3) positive 4T1 cells normalized to average sphere size in each group. 4T1 spheres were co-cultured with T-cells for 72 hours following 24-hour pretreatment with vehicle or 1 μM VT107 (Mean±SD, student t-test). Representative IHC images are shown (scale bar is 10 μm). Panel D shows RTCA of 4T1 cells measured by electric impedance sensing of attached cells (cell index) over time. Jurkat cells that harbor shControl or shTEAD were added to the cancer cells to monitor their cytotoxicity (Mean±SD, student t-test). Panel E shows volcano plot of the altered gene expression in Jurkat cells that harbor shControl or shTEAD. Circles in bracketed sections indicate regulated genes with −Log p-value >1.3 and log 2FC|=2. Panel F shows IPA depicting altered signaling pathways in shTEAD versus shControl Jurkat cells that have a p-val <0.05.

### DETAILED DESCRIPTION OF THE INVENTION

The practice of the present invention can employ, unless otherwise indicated, conventional techniques of pharmaceutics, formulation science, tissue biology, cell biology, oncology immunology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” preceding a numerical value includes ±10% of the recited value. For example, a concentration of about 1 mg/mL includes 0.9 mg/mL to 1.1 mg/mL. Likewise, a concentration range of about 1% to 10% (w/v) includes 0.9% (w/v) to 11% (w/v).

An “active agent” is an ingredient that is intended to furnish biological activity. The active agent can be in association with one or more other ingredients.

An “inhibitor” is an ingredient that is intended to furnish an inhibitory effect on biological activity. The inhibitor can be in association with one or more other ingredients.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline.

A “subject” or “individual” or “animal” or “patient” or “mammal,” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc. In some embodiments the subjects are human.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the patient shows total, partial, or transient alleviation or elimination of at least one symptom or measurable physical parameter associated with the disease or disorder.

A “control population” or a “population of control patients” is a group of subjects that have not received treatment. Subjects in the control population have the same disease or disorder as the subject being compared to the control population. For example, a clinical outcome of a cancer patient receiving a pharmaceutical composition or method of the invention is compared with the average (median) outcome of subjects having the same type of cancer who did not receive a pharmaceutical composition or method of the invention.

The terms “inhibit,” “block,” and “suppress” are used interchangeably and refer to any statistically significant decrease in occurrence or activity, including full blocking of the occurrence or activity. For example, “inhibition” can refer to a decrease of about 10%, 20%, 30%, 40%, 50%, 60%, 70%, 80%, 90% or 100% in activity or occurrence. An “inhibitor” is a molecule, factor, or substance that produces a statistically significant decrease in the occurrence or activity of a process, pathway, or molecule.

The terms “induce” or “promote” refers to an act of causing or generating, either directly or indirectly, a prescribed result.

A “tumor” or “solid tumor” is a mass of neoplastic cells, such as cancer cells. The terms “advanced,” “metastatic,” and “advanced/metastatic” are used interchangeably to describe a cancer in which malignant cells have migrated from the original tumor to another location, for example, another organ, in a patient's body.

“Primary cancer” refers to the original cancer that developed in the body (in an organ or tissue). Similarly, a “primary tumor” is the original tumor where cancers cells initially developed and formed a mass. “Primary cancer cells” refers to cancer cells of the primary cancer.

“Metastatic cancer” or “secondary cancer” refers to cancer developed at a site anatomically separated from the original site at which the primary cancer developed, due to metastasis of the primary cancer. The site of the secondary cancer may be a different tissue, different organ, or a different area of the same tissue or same organ, from that of the primary cancer. “Metastatic tumor” or “secondary tumor” is a tumor formed by the metastasis of the primary cancer at a site anatomically separated from the site of the primary tumor. “Secondary cancer cells” refers to cancer cells of the secondary cancer. Populations of secondary cancer cells may also be referred to as “metastases.”

““Alveolar macrophage” refers to a type of macrophage that is found in the airways and at the level of the alveoli in the lungs. They are responsible for removing particles such as dust or microorganisms from the respiratory surfaces, and play a critical role in homeostasis, host defense, and tissue remodeling.

“Infiltrating monocyte/macrophage” refers to a type of monocyte that is recruited into tissues when an inflammatory reaction occurs and differentiate into a macrophage. Tumor infiltrating macrophages can enhance angiogenesis and increase production of factors that can promote tumor growth and metastasis.

“Polarize,” “polarization,” and the like, refers to a process by which macrophages adopt different functional programs. Often, polarization occurs in response to signals from the macrophages' microenvironment. Polarization results in a macrophage having a phenotype based on its secretory profile, gene expression, and/or function.

“M1 macrophage,” also known as a “classically activated” macrophage or a “tumor-associated” macrophage, is a polarized macrophage that is generally inflammatory and anti-tumor. Examples of markers for an M1 macrophage include CD11c, CD86, HLA-DRA, and phospho-STAT1 (Tyr701).

“M2 macrophage,” also known as a “alternatively activated” macrophage, is a polarized macrophage that is generally anti-inflammatory and pro-tumor. Examples of markers for an M2 macrophage include CD163 and CD206.

### TEAD and TEAD Inhibitors

The methods and compositions provided by the present invention involve TEAD inhibitors.

TEAD, or transcriptional enhanced associate domain, refers to a family of transcription factors that are broadly expressed in most tissues. TEADs are the final nuclear effectors of the Hippo pathway, which regulate cell growth, proliferation, and tissue homeostasis, and plays a key role in organ size control and tumor suppression. YAP, and the homologous oncoprotein TAZ, interact with TEAD to promote cell growth and proliferation and inhibit apoptosis.

The TEAD family is comprised of four conserved homologues, identified as TEAD1, TEAD2, TEAD3, and TEAD4. Each TEAD has tissue-specific roles during embryonic development; for example, TEAD1 is involved in cardiogenesis (Chen et al., 1994), TEAD2 plays a role in neural development (Kaneko et al., 2007), and TEAD4 helps determine trophectoderm lineage (Yagi et al., 2007). TEADs also have highly similar domain architectures, as the TEAD N-terminus share a highly conserved 68-amino acid TEA/ATTS DNA-binding domain (Jacquemin et al., 1996). Further, TEAD activity relies primarily on the C-terminus, in which all TEADs share their transactivation domain in order to recruit transcriptional coactivators such as YAP/TAZ, corepressors such as VGLL1-4, and chromatin remodeling factors such as NuRD (Huh et al., 2019).

Inhibitors of TEAD may prevent or reduce TEAD activity. TEAD inhibitors in accordance with the present invention may be, for example, in the form of a small molecule; an antagonist of TEAD such as an antibody or fragment thereof, a binding protein, a polypeptide, and any combination thereof, that targets TEAD; or a nucleic acid molecule, such as a double stranded ribonucleic acid (dsRNA), small hairpin RNA or short hairpin RNA (shRNA), small interfering RNA (siRNA), or antisense RNA that targets TEAD.

In some embodiments, the TEAD inhibitor is an inhibitor of TEAD auto-palmitoylation. Each of the TEADs requires auto-palmitoylation on the sulfhydryl of a conserved cysteine to become functional. (Chan et al., 2016; Noland et al., 2016).

In some embodiments, the TEAD inhibitor prevents TEAD binding with other molecules at the C-terminus. For instance, the YAP-binding domain of TEAD has a hydrophobic central pocket that can be targeted to block the interaction of TEAD with YAP (Pobbati et al., 2015). A TEAD inhibitor that binds to the central pocket of the YAP-binding domain of TEAD is flufenamic acid.

Examples of TEAD inhibitors for use in the present invention include, but are not limited to, VT101, VT102, VT103, VT104, VT105, VT107, flufenamic acid, TED-347, Super-TDU, zoledronic acid, niflumic acid, MGH-CP-1, quinolinol analogs Q2, DC-TEADinO2, K-975, MYF-01-037, kojic acid analogue 19, protoporphyrin IX. Verteporfin, TM2, IK-930, OPN-9840, and digitoxin (see WO2019040380; WO2020097389; Holden et al., 2020; Lu et al., 2021; Tang et al., 2021; Pobatti et al., 2015; Jiao et al., 2014; Bum-Erdene et al., 2019; Lu et al., 2019; Kurppa et al., 2020; Kaneda et al., 2020; Tang et al., 2021; Li et al., 2020; Hu et al., 2022; Chen et al., 2024; Tolcher et al., 2022).

In some embodiments the present invention provides combinations comprising one or more of the inhibitors described herein. The combinations may be for use in the methods of treatment described herein.

In some embodiments the present invention provides pharmaceutical compositions comprising one or more of the inhibitors described herein and a pharmaceutically-acceptable carrier. As used herein, a “pharmaceutically-acceptable carrier” is, or comprises, a substance that is useful in preparing a composition suitable for administration to a living subject (such as a living human subject) and that is generally safe and non-toxic. Suitable pharmaceutically-acceptable carriers may be, or may comprise, a saline solution (e.g., a phosphate buffered saline solution), water, an emulsion (such as an oil/water or water/oil emulsion), a wetting agent, a diluent, a filler, a salt, a buffer, a stabilizer, a solubilizer, a lipid, or any other substance known in the art for use in preparing a composition suitable for administration to a living subject. The pharmaceutical compositions may be for use in the methods described herein.

Methods of preparing pharmaceutical compositions are well-known to those of ordinary skill in the art, or can be readily determined by those of ordinary skill in the art. For example, the pharmaceutical compositions can be prepared for oral administration and therefore are in oral dosage forms include, e.g., capsules, tablets, aqueous suspensions, and solutions. Nasal aerosol or inhalation dosage forms can be prepared, for example, as solutions in saline, employing benzyl alcohol or other suitable preservatives, absorption promoters to enhance bioavailability, and/or other conventional solubilizing or dispersing agents.

### Methods of Use

The present invention provides various methods involving TEAD inhibitors and/or pharmaceutical composition thereof, as described herein.

Thus, in one aspect, the present invention provides a method of treating secondary cancer in the lung of a subject in need thereof. The method comprises administering a pharmaceutical composition comprising an effective amount of a TEAD inhibitor to the subject. In some embodiments, the present invention provides a method of reducing the rate of growth of a secondary cancer tumor, or provides a method of reducing the rate of growth of secondary cancer cells, or provides a method of inhibiting the growth of a secondary cancer tumor, or provides a method of inhibiting the growth of secondary cancer cells, or provides a method of halting the growth of a secondary cancer tumor, or provides a method of halting the growth of secondary cancer cells, or provides a method of reducing the size of a secondary cancer tumor, or provides a method of reducing the number of secondary cancer cells, or provides a method of causing regression of a secondary cancer tumor, or provides a method of causing the regression of secondary cancer cells, or provides a method of reducing the grade of a secondary cancer tumor, or provides a method of eliminating a secondary cancer tumor, or provides a method of eliminating secondary cancer cells; these method comprising administering to the subject a pharmaceutical composition comprising an effective amount of a TEAD inhibitor to the subject.

In another aspect, the present invention provides a method of increasing number of T-cells at a secondary tumor or at the site of secondary cancer cells in a lung of a subject in need thereof. The method comprises administering a pharmaceutical composition comprising an effective amount of a TEAD inhibitor to the subject. In some embodiments, the T-cells are CD8+ T-cells.

In yet another aspect, the present invention provides a method of increasing number of alveolar macrophages in the lung of a subject in need thereof. In yet another aspect, the present invention method provides a method of decreasing number of infiltrating monocytes/macrophages in the lung of a subject in need thereof. These methods comprise administering a pharmaceutical composition comprising an effective amount of a TEAD inhibitor to the subject. In some embodiments, the subject is at risk of developing a secondary tumor in the lung, for example, the subject has primary cancer and/or primary tumor; or the subject has a cancer susceptibility gene such as BRCA2, APC, RB1, PAX8, CDKN2A, GSTP1, CASP8, TP53, TERT, CLPTM1L, FAS, ALDH2, GSTM1, SULTIA1, NQO1, ACE, XRCC1, or CYP1B1.

In a further aspect, the present invention provides a method of reducing lung metastases in a subject in need thereof, the method comprising administering a pharmaceutical composition comprising an effective amount of a TEAD inhibitor to the subject. In some embodiments, the subject may have metastatic cancer of the lung.

In another aspect, the present invention provides a method of inducing T-cell activation in the lung of a subject with lung cancer. In yet another aspect, the present invention provides a method of activating IL12 signaling in the lung of a subject with lung cancer. These methods comprise administering a pharmaceutical composition comprising an effective amount of a TEAD inhibitor to the subject. In some embodiments, the lung cancer is a metastatic cancer.

In another aspect, the present invention provides a method of inducing polarization of one or more M2 macrophages to M1 macrophages in the lung of a subject with lung cancer. The method comprises administering a pharmaceutical composition comprising an effective amount of a TEAD inhibitor to the subject.

In some embodiments, the subject comprises primary cancer and/or has a primary tumor in non-lung tissue. Examples of non-lung tissue of the primary cancer and/or primary tumor includes, but is not limited to breast, bone, esophagus, colon, rectum, kidney, cervix, prostate, larynx, liver, pancreas, brain, and skin. In some embodiments, the subject comprises primary cancer and/or has a primary tumor in lung tissue.

In some embodiments, the primary cancer may be of the gastrointestinal tract (e.g., colon carcinoma, rectal carcinoma, colorectal carcinoma, colorectal cancer, colorectal adenoma, small and/or large bowel carcinoma, esophageal carcinoma, stomach carcinoma, pancreatic carcinoma, pancreatic endocrine tumors), endometrial carcinoma, dermatofibrosarcoma protuberans, gallbladder carcinoma, biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, nasopharyngeal cancer, breast carcinoma (e.g., ductal breast cancer, luminal breast cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B-cell, Burkitt, cutaneous T-cell, histiocytic, lymphoblastic, T-cell, thymic), gliomas, adenocarcinoma, adrenal tumor, hereditary adrenocortical carcinoma, brain malignancy (tumor), glioblastoma (e.g., multiforme, astrocytoma), glioma, head and neck cancer, hepatoma, leukemia, lymphosarcoma, melanoma, mammary tumor, mastocytoma, medulloblastoma, mesothelioma, monocyte tumor, multiple myeloma, myelodysplastic syndrome, myeloma, nephroblastoma, nervous tissue glial tumor, nervous tissue neuronal tumor, neurinoma, neuroblastoma, neuroendocrine tumors, oligoastrocytoma, oligodendroglioma, osteochondroma, osteomyeloma, osteosarcoma (e.g., Ewing's), papilloma, pituitary tumor, plasmacytoma, retinoblastoma, rhabdomyosarcoma, sarcoma (e.g., Ewing's, gastric, histiocytic cell, Jensen, osteogenic, reticulum cell), schwannoma, subcutaneous tumor, testicular tumor, thymoma, and trichoepithelioma. In certain embodiments, the primary cancer is triple negative breast cancer.

In carrying out the methods described herein, any suitable method or route of administration can be used to deliver the pharmaceutical composition described herein. The term “administration” includes any route of introducing or delivering the pharmaceutical composition to subjects. In some embodiments the pharmaceutical composition is administered systemically. In some embodiments the pharmaceutical composition is administered locally. “Systemic administration” refers to introducing or delivering to a subject a specified composition via a route which introduces or delivers the composition to extensive areas of the subject's body (e.g., greater than 50% of the body), for example through entrance into the circulatory or lymph systems. By contrast, “local administration” refers to introducing or delivering to a subject a specified composition via a route which introduces or delivers the TEAD inhibitor to the area or area immediately adjacent to the point of administration and does not introduce the TEAD inhibitor systemically in a therapeutically significant amount. For example, a locally administered TEAD inhibitor is easily detectable in the local vicinity of the point of administration, but is undetectable or detectable at negligible amounts in distal parts of the subject's body.

In some embodiments, administration can be carried out by any suitable route known in the art, including intratumoral, intravenous, subcutaneous, oral, topical, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. Administration includes self-administration and administration by another. The suitability of a given route or means of administration can be readily determined by a physician.

The efficacy of a given pharmaceutical composition or method of the present invention can be demonstrated or assessed using standard methods known in the art, such as magnetic resonance imaging (MRI), x-radiographic imaging, computed tomographic (CT) scan, flow cytometry or fluorescence-activated cell sorter (FACS) analysis, histology, gross pathology, blood chemistry, marker analysis, and gene expression, including but not limited to changes detectable by ELISA, ELISPOT, RIA, chromatography, and the like. Further, the subject undergoing therapy with the TEAD inhibitor can experience improvement in the symptoms associated with the disease being treated. In some embodiments, a comparison can be made of e the efficacy of a given/“test” composition or method to a “control” composition or method. For example, the efficacy of a given composition or method in treating a secondary tumor may be demonstrated or assessed by comparing its ability to improve one or more clinical indicators or symptoms of a tumor as compared to that of a control composition or control method, such as a placebo control. For instance, a comparison can be made between different subjects (e.g., between a test group of subjects or a control group of subjects). Similarly, the efficacy of a given composition or method in treatment can be demonstrated or assessed in a single subject by comparing that subject's tumor before and after treatment.

In some embodiments, the pharmaceutical composition comprising a TEAD inhibitor may be administered with one or more different active agents, i.e., active agents that are different from the TEAD inhibitor. For instance, the different active agent may be an immune modulator, such as an immune checkpoint inhibitor such as CTLA-4 inhibitors, PD-1 inhibitors, PD-L1 inhibitors, LAG-3 inhibitors, or TIGIT inhibitors; cytokine; chimeric antigen receptor (CAR) T-cell therapy; cancer vaccine; small molecule immune modulators such as sirolimus, tacrolimus, or thalidomide derivatives; kinase inhibitors such as dasatinib; or other active agents that impact B-cell receptor (BCR) signaling that are known in the art.

Where the TEAD inhibitor and one or more different active agents are administered, they may be administered simultaneously, sequentially, or at overlapping times. Similarly, where the TEAD inhibitor and one or more different active agents are administered, they may be administered together in the same composition or separately in different compositions. As used herein, the term “separate” or “separately” in the context of a therapeutic use refers to an administration of at least two active ingredients at the same time or at substantially the same time by different routes. As used herein, the term “sequential” or “sequentially” in the context of a therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case. Further, as used herein, the term “simultaneous” or “simultaneously” in the context of a therapeutic use refers to the administration of at least two active ingredients by the same route and at the same time or at substantially the same time.

The methods involve administering a pharmaceutical composition comprising an effective amount of a TEAD inhibitor. As used herein the term “effective amount” refers to an amount of a TEAD inhibitor that is sufficient to achieve, or contribute towards achieving, one or more of the outcomes described herein. An appropriate “effective” amount in any individual case may be determined using standard techniques known in the art, such as dose escalation studies, and may be determined taking into account such factors as the desired route of administration (e.g., systemic vs. local), the desired frequency of dosing, etc. Furthermore, an “effective amount” may be determined in the context of any co-administration to be used. One of skill in the art can readily perform such dosing studies (whether using TEAD inhibitors or TEAD inhibitors in combination with other agents) to determine appropriate doses to use, for example, using assays that may administration of the TEAD inhibitors described herein single or in combination with other agents to subjects—such as animal subjects routinely used in the pharmaceutical sciences for performing dosing studies.

For example, in some embodiments the dose of a TEAD inhibitor of the invention may be calculated based on studies in humans or other mammals carried out to determine efficacy and/or effective amounts of the TEAD inhibitor. The dose amount and frequency or timing of administration may be determined by methods known in the art and may depend on factors such as pharmaceutical form of the active agent, route of administration, whether only one TEAD inhibitor is used or a TEAD inhibitor and another active agent (for example, the dosage of the TEAD inhibitor required may be lower when used in combination with a second active agent), and patient characteristics including age, body weight, or the presence of any medical conditions affecting drug metabolism.

In some embodiments suitable doses of the TEAD inhibitors described herein can be determined by performing dosing studies of the type that are standard in the art, such as dose escalation studies.

Dosing regimens can also be adjusted and optimized by performing studies of the type that are standard in the art. In some embodiments the TEAD inhibitor is administered daily, or twice per week, or weekly, or every two weeks, or monthly.

In certain embodiments the compositions and methods of treatment provided herein may be employed together with other compositions and treatment methods known to be useful for tumor therapy, including, but not limited to, surgical methods (e.g., for tumor resection), radiation therapy methods, treatment with chemotherapeutic agents, treatment with antibodies, treatment with immunotherapeutic agents, treatment with cell therapy methods, treatment with tyrosine kinase inhibitors, and the like. Similarly, in certain embodiments the methods of treatment provided herein may be employed together with procedures used to monitor disease status/progression, such as biopsy methods and diagnostic methods (e.g., MRI, X-ray, PET, or CAT methods, or other imaging methods).

For example, in some embodiments the methods described herein and/or the TEAD inhibitors and compositions described herein may be employed or administered to a subject prior to performing surgical resection of a tumor, for example in order to shrink a tumor prior to surgical resection. In other embodiments the methods described herein and/or the TEAD inhibitors and compositions described herein may be employed or administered to a subject both before and after performing surgical resection of a tumor.

### EXAMPLE

### Example 1: Effect of TEAD Inhibition on Lung Metastasis

The effect of TEAD inhibition on lung metastasis was studied on non-obese diabetic (NOD) mice with severe combined immunodeficiency (SCID).

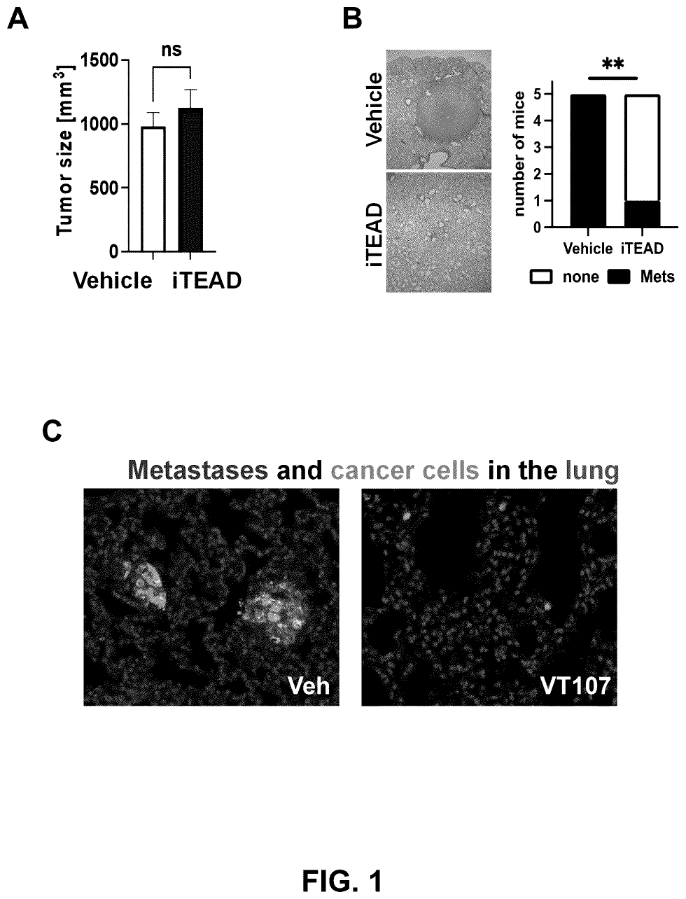

Parental ductal carcinoma in situ (DCIS) was mixed with genetically-modified DCIS cells expressing AIB1-Δ4 (“DCIS-Δ4”) (see Sharif et al., 2021) at a 4:1 ratio, and the mix was injected into the mammary fat pad (MFP) of the mice. One group of mice received 30 mg/kg of the TEAD inhibitor VT107 (“iTEAD”) and the other group received vehicle treatment by daily oral gavage for 4 weeks. The primary tumors were removed by survival surgery. There was no difference observed in the growth rate or final volume of the primary tumors. However, iTEAD-treated mice did not develop lung metastases compared to the vehicle-treated group (FIG. 1, Panels A and B). iTEAD-treatment halted outgrowth from cancer cells that had disseminated to the lungs, but were unable to manifest into over metastases (FIG. 1, Panel C).

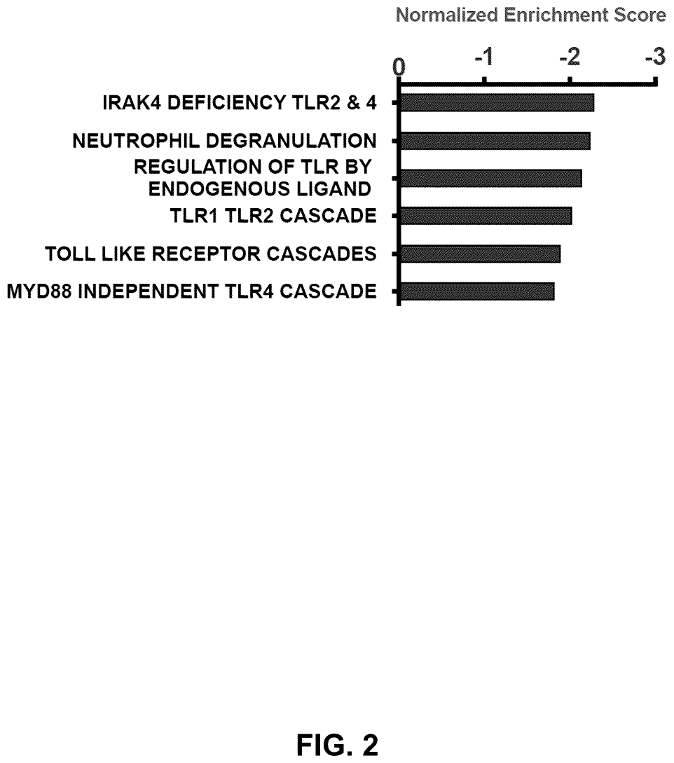

Lung tissues were subjected to RNA-Seq analysis, and Gene Set Enrichment Analysis (GSEA) was used to determine altered signaling pathways upon iTEAD treatment. Gene expression analysis of lung tissue revealed a drop in Toll-like receptor 4 (TLR4) signaling pathway upon iTEAD treatment (FIG. 2).

### Example 2: Effect of TEAD Inhibition in a Syngeneic Mouse Model

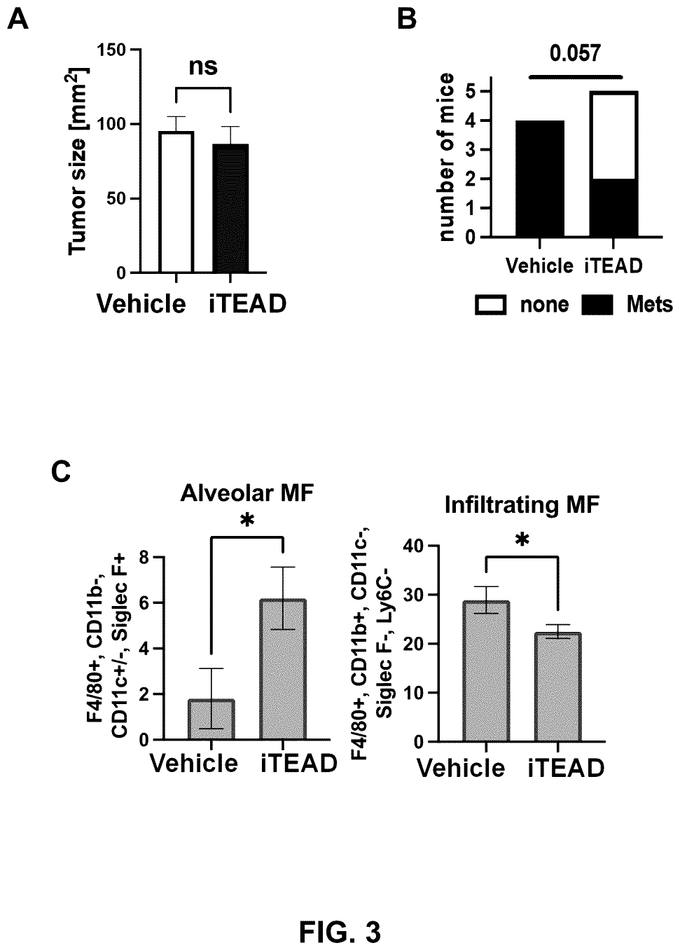

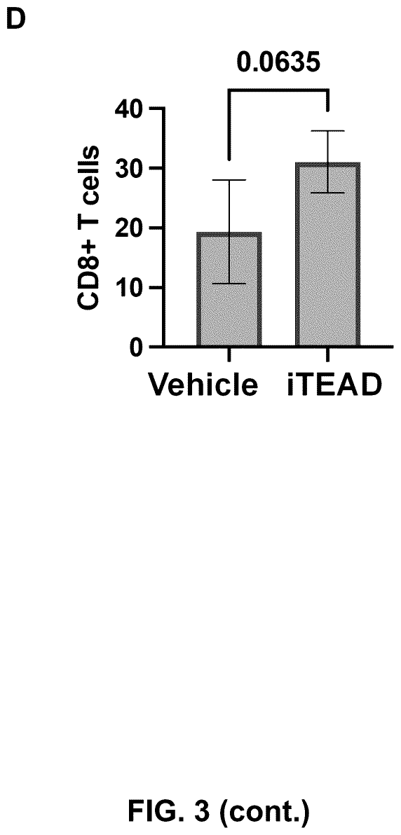

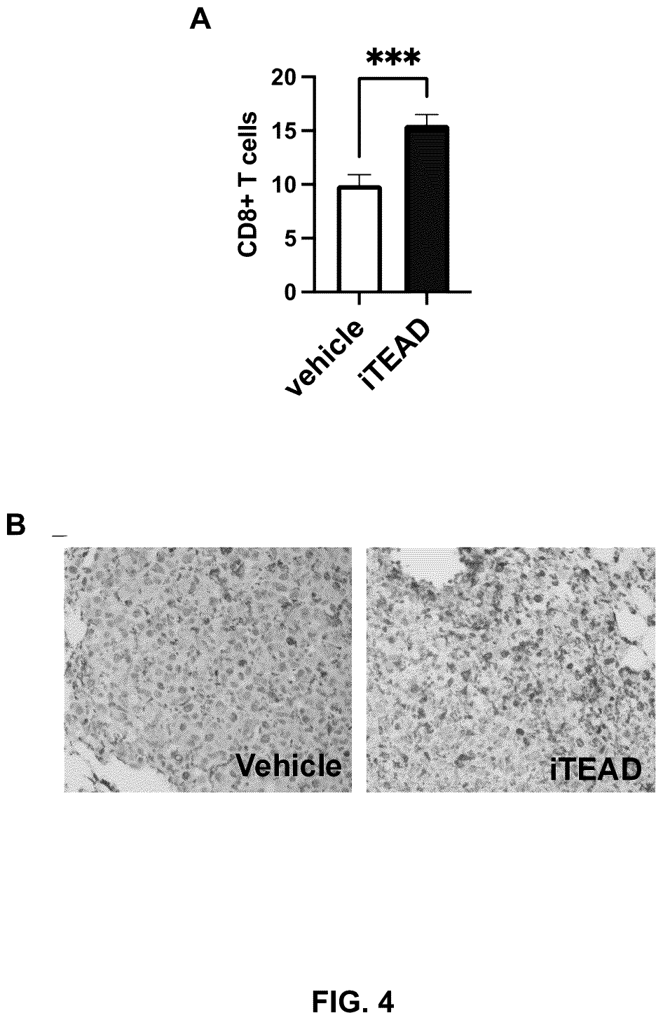

A study was conducted to determine whether an anti-metastatic effect was observed in a syngeneic mouse model. In this study, E0771 mammary carcinoma cells were injected into the MFP of C57/BL6 mice. Treatment for this group started after tumors were palpable at 3-4 weeks. Similarly, no effect was observed on the primary tumor growth, yet lung metastases were decreased (FIG. 3, Panels A and B). Immune profiling of tissues by flow cytometry revealed a shift in monocyte/macrophage subpopulations, namely an increase in resident alveolar macrophages (AlvMF) and a decrease in infiltrating macrophages (InfMF) in the lungs (FIG. 3, Panel C). Additionally, lung infiltrating CD8+ T-cells increased in iTEAD-treated mice (FIG. 3, Panel D). CD8+ immunohistochemistry of lung tissue showed an increase in the number and infiltration of CD8+ cells into micro-metastases in the iTEAD versus control group (FIG. 4). These results suggest that iTEAD-modulated microenvironment-perhaps through an alternative macrophage phenotype—may have reversed cytotoxic T-cells exclusion from the lungs as seeded cancer cells grow to form metastases, which is significant given the crosstalk between macrophages and T-cells during the elimination of malignant cells.

Together, this study suggests that TEAD can have a role in regulating resident and infiltrating monocyte/macrophage phenotypes in the lungs that can impact metastatic growth.

### Example 3: Effect of iTEAD on Lung Immune Microenvironment at Steady State

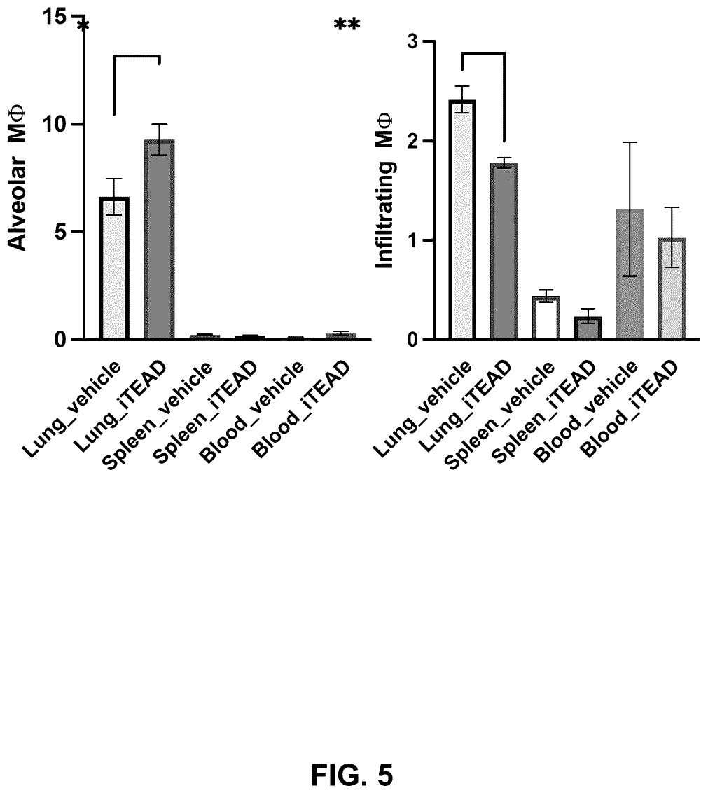

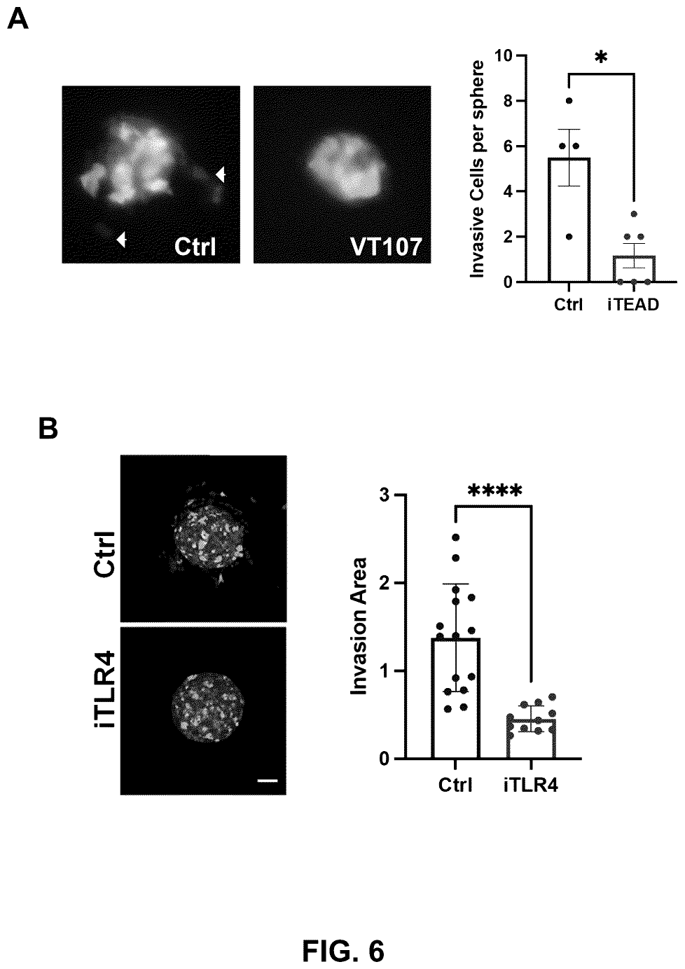

To determine iTEAD effect on lung immune microenvironment at steady state, naïve C57/BL6 mice were treated with iTEAD. The results showed a robust increase in AlvMF and decrease in infiltrating monocytes/macrophages in the lungs, but not in other tissues like spleen or blood (FIG. 5). In the absence of cancer cells in these naïve mice, no change in CD8+ T-cells was observed between treatment groups. These results suggest that the increase in CD8+ T-cells in iTEAD-treated mice may be tumor-specific (FIG. 3, Panel D). On the cancer cell front, TEAD inhibition had no effect on proliferation yet reduced migration and 3D spheroid collective invasion of breast cancer cells in vitro. A similar phenotype was seen with a TLR4 inhibitor, suggesting a feedback loop along the TLR4/TEAD signaling axis in cancer cells (FIG. 6).

### Example 4: Effect of TEAD-Inhibited Macrophages on T-Cell Behavior

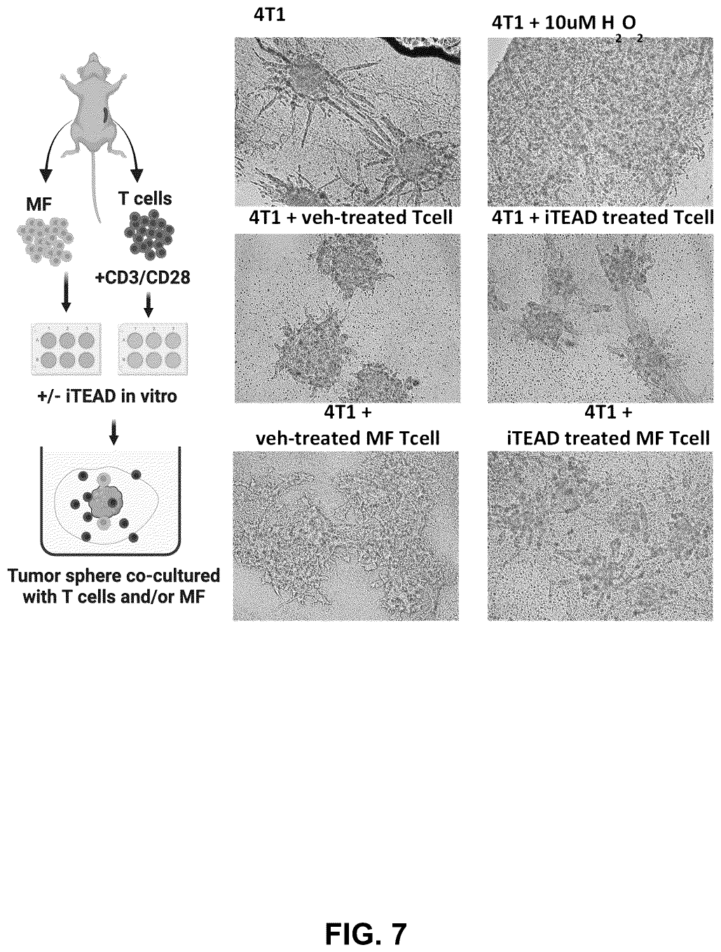

Based on the results of Examples 2 and 3, CD8+ T-cells in iTEAD-treated lungs was increased only in the presence of tumors but not in naïve mice (see FIG. 3). To determine if a change in T-cell behavior is directly related to TEAD-inhibited macrophages, an ex vivo 3D coculture system to monitor cancer cell killing by T-cells (FIG. 7).

T-cells were isolated from the spleens of naïve mice, activated by CD3/CD28, and treated with either 1 μM iTEAD or vehicle. Peritoneal macrophages were isolated from the peritoneal lavage of naïve mice after attachment on culture dishes and treated with either 1 μM iTEAD or vehicle. The immune cells were mixed with tumor cells that were aggregated into spheres in u-shaped molds for 24 hours (FIG. 7). Tumor cell killing was observed in wells with T-cells, and to a higher extent with iTEAD treatment and in the presence of iTEAD-treated macrophages. These results indicate that the presence of iTEAD-treated macrophage increased T-cell activity.

### Example 5: Effect of TEAD-Inhibition by RNAi on Macrophages and T-Cell Behavior

Short hairpin (sh) RNAi expression vectors or synthetic RNAi molecules are used to knockdown TEAD levels in macrophages and T-cells.

Macrophage and T-cell line models, such as THP1 and Jurkat cells, are used for in vitro studies to knockdown TEAD protein expression levels using RNAi. For example, a lentivirus with small hairpin RNA (shRNA) targeting a common region on multiple TEAD family members is used for infection of the cell lines. A nontargeting shRNA is used as a control. After confirmation of TEAD knockdown (KD), change in macrophage activation markers (M1=pro-inflammatory vs M2=anti-inflammatory) are examined by qPCR and cytokines arrays while T-cells are stimulated by CD3/CD28. Change in CD107, a degranulation marker, is examined flow cytometry.

### Example 6: Effect of TEAD-Inhibition by RNAi on Macrophages and T-Cell Behavior

An investigation was performed on the immune stromal effects of TEAD inhibition on the metastatic niche of the lungs.

### Results

### TEAD Inhibition Alters the Lung Immune Microenvironment and Activates IL12 Signaling

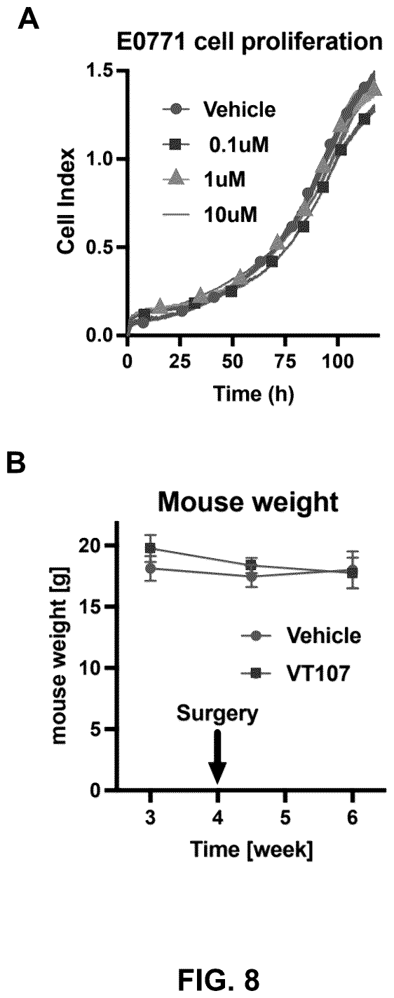

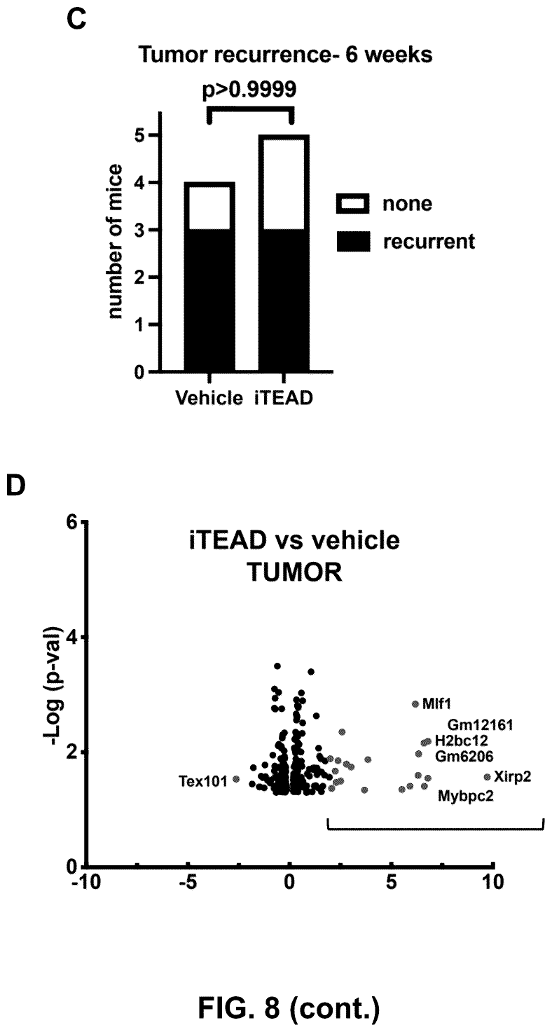

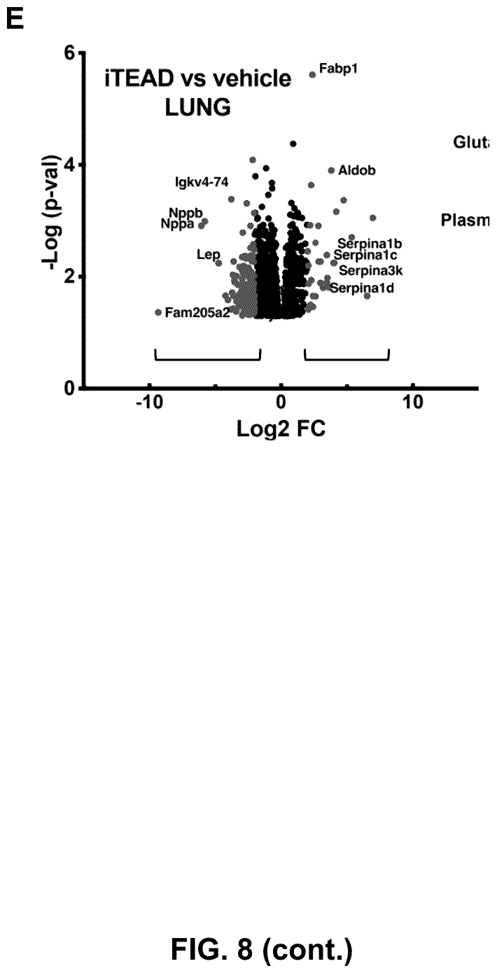

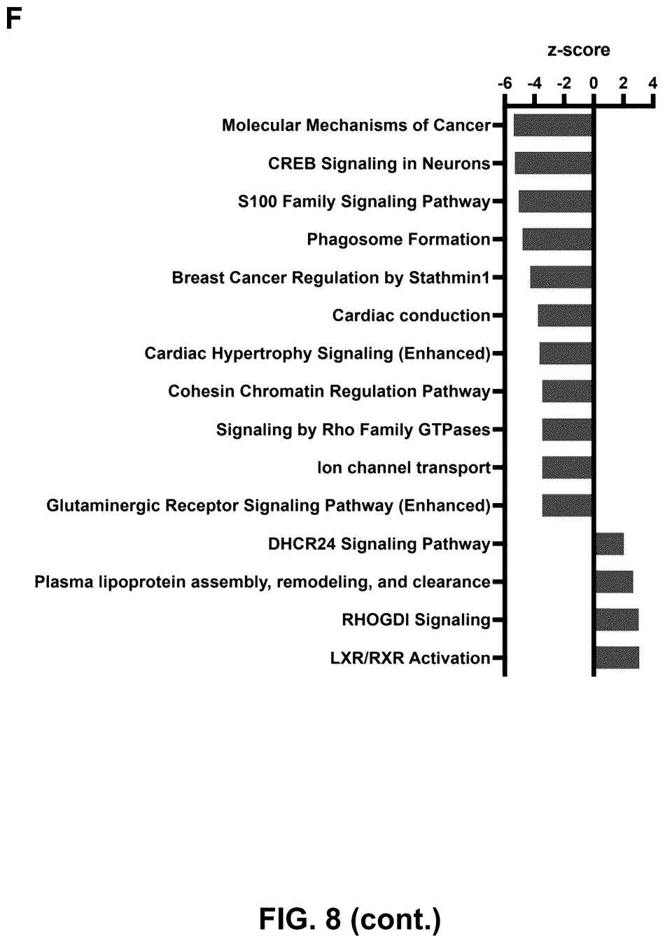

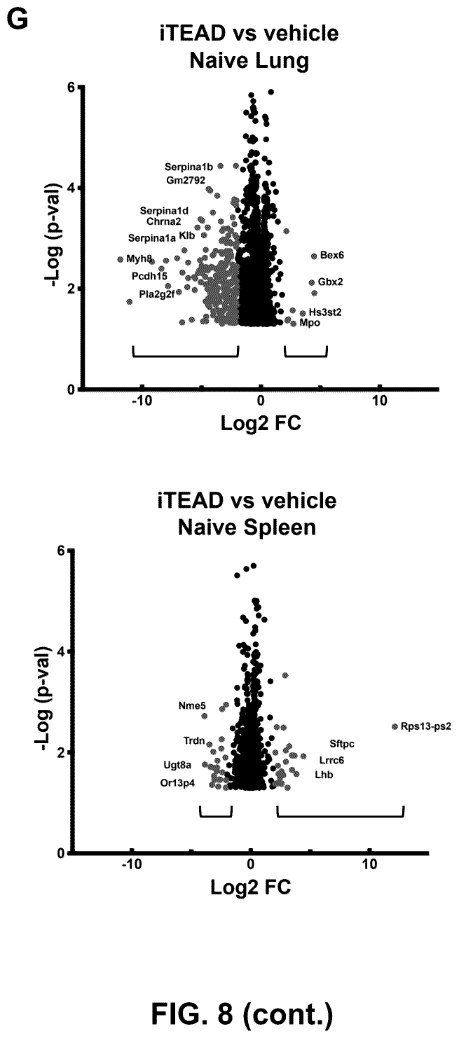

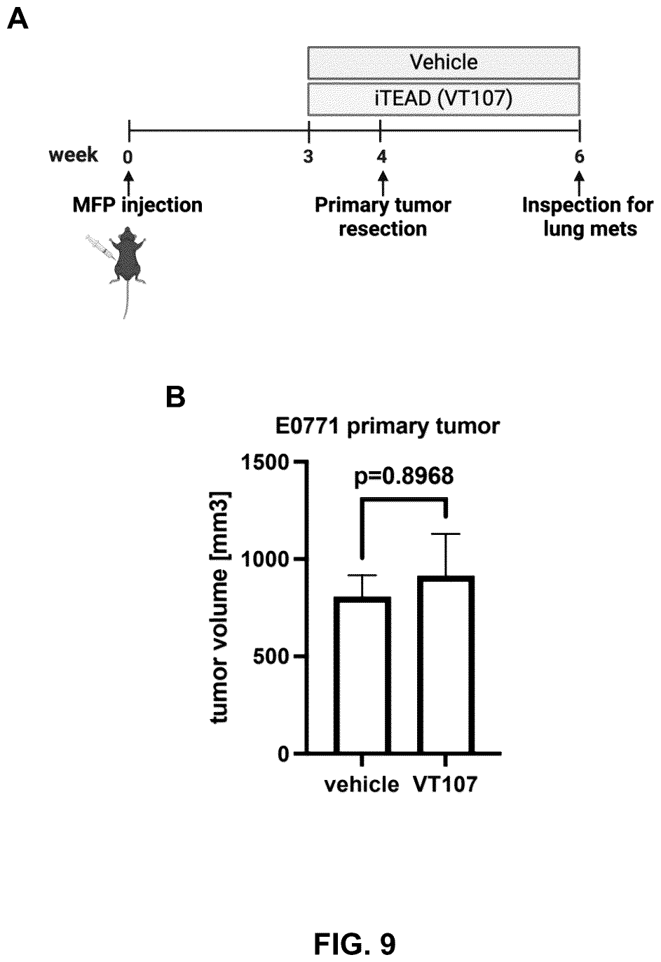

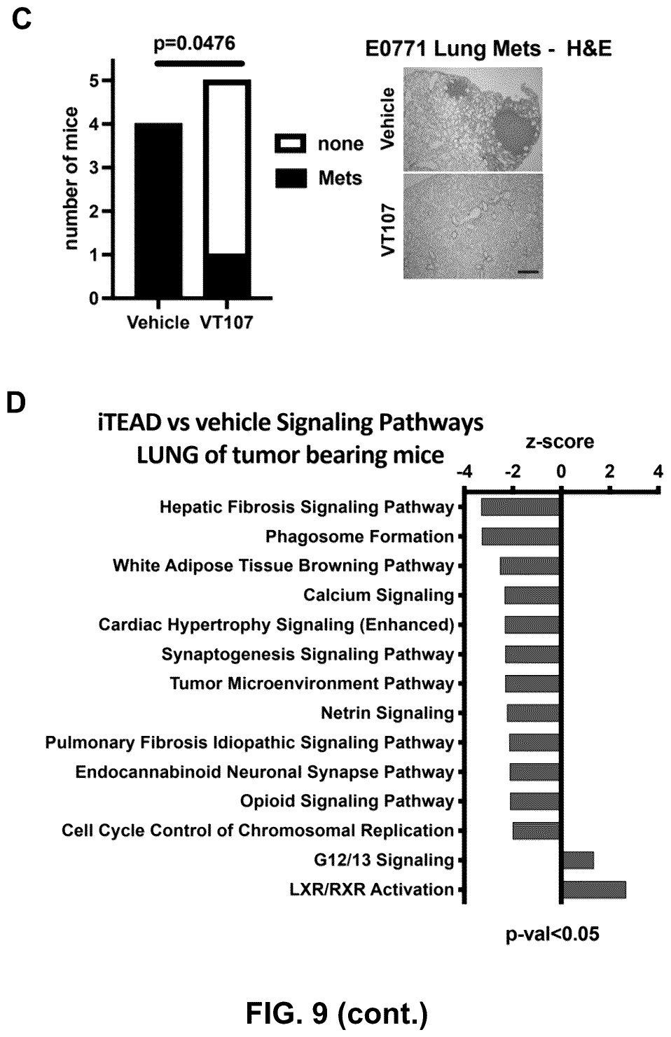

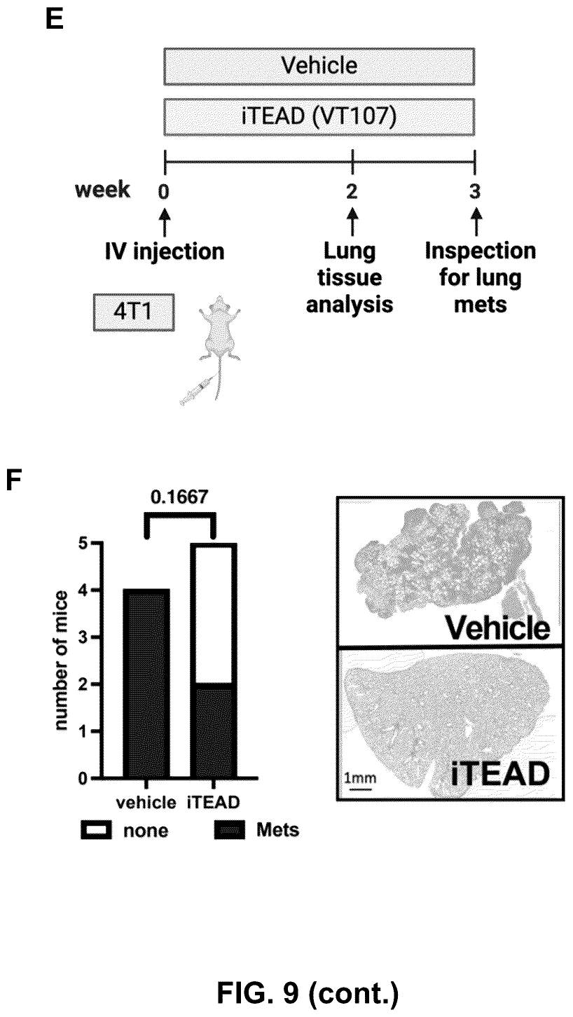

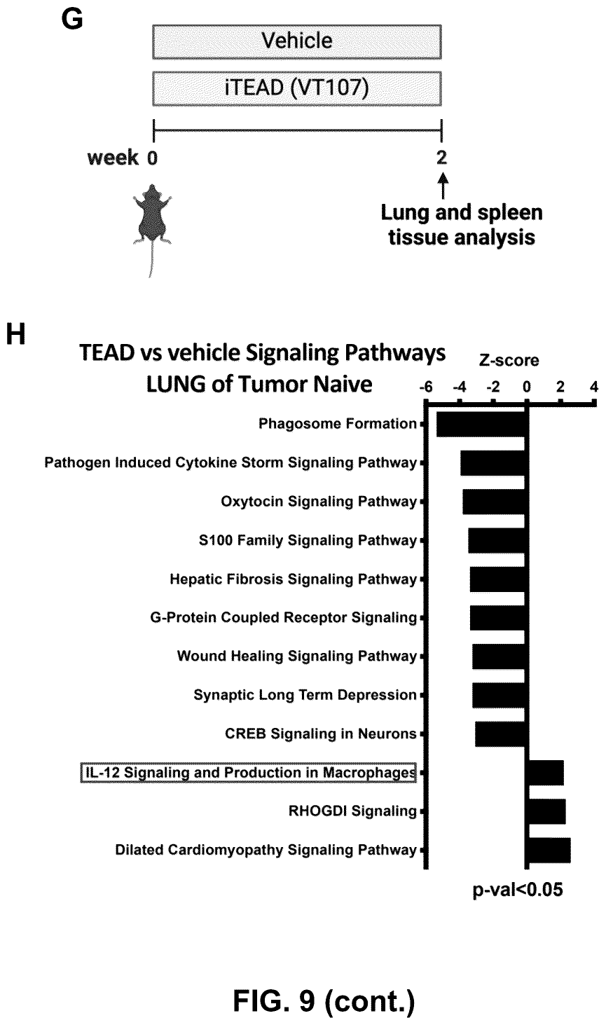

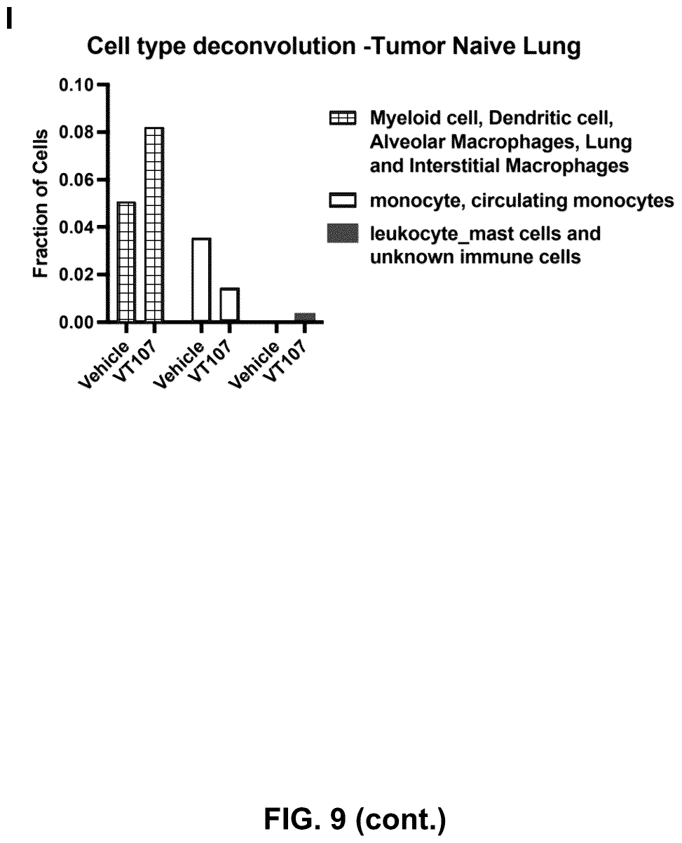

To determine extrinsic effects of YAP/TEAD signaling on the lung microenvironment of the metastatic niche, a pan TEAD inhibitor, VT107 (Tang et al., 2021) was used that did not impact the proliferation of the mammary carcinoma cell line E0771 in vitro (FIG. 8, Panel A). E0771 cells were injected into the mammary fat pad of C57BL/6 mice (Johnstone et al., 2015). When primary tumors became palpable at 3 weeks, mice were treated daily with VT107 or vehicle control. Mice weights were monitored throughout treatment (FIG. 8, Panel B). Primary tumors were surgically resected at week 4 and mice were euthanized at week 6 to inspect lungs for metastases (FIG. 9, Panel A)

There was no difference in the primary tumor size between treatment groups nor the size of recurrent tumors at 6 weeks (FIG. 9, Panel B and FIG. 8, Panel C). Despite the early termination of the experiment due to recurrent mammary tumors, histopathological assessment of lung sections from VT107-treated mice showed significantly less incidence of spontaneous metastases compared to the vehicle treated group (FIG. 9, Panel C). To investigate tissue specific effects, gene expression changes between treatment groups in lung and tumor tissues were analyzed by RNA-sequencing. A higher number of gene changes due to treatment was observed in the lungs compared to the primary tumors (FIG. 8, Panels D and E). Pathway analysis showed a decrease in disease-related inflammation pathways such as hepatic and lung fibrosis and cardiac hypertrophy, while LXR/RXR, an anti-inflammatory pathway, was increased in the lung tissues (FIG. 9, Panel D). To reduce the impact of TEAD inhibition on cancer cell intrinsic function such as dissemination and invasion, 4T1, a triple negative mouse mammary carcinoma cell line (Pulaski & Ostrand-Rosenberg, 2000), was injected into the tail vein of BALB/c mice that directly seed into the lung tissue. A reduction in lung metastases, as well as the suppression of inflammatory pathways, were observed (FIG. 9, Panels E and F, and FIG. 8, Panel F).

To investigate how TEAD inhibition influences the immune microenvironment in the normal lung, C57BL/6 mice were treated with 30 mg/kg of VT107 or vehicle control via oral gavage for 2 weeks. Lung, spleen and blood tissues were harvested for immune cell analysis and RNA-sequencing (FIG. 9, Panel G). Gene expression analysis of the lungs showed a significantly higher number of downregulated genes compared to upregulated genes while there were minimal changes in the spleen (FIG. 8, Panel G). Ingenuity pathway analysis of the lung gene expression data revealed overall reduction in inflammation with a decrease in cytokine storm, S100 family, wound healing and fibrosis signaling pathways, however, there was an increase in the IL12 signaling pathway (FIG. 9, Panel H) which has been implicated in antitumor inflammation (Kaczanowska et al., 2021; Xue et al., 2022). RNA-seq data was analyzed by Cibersortx (Chen et al., 2018) to deconvolute the abundance of immune cell types in the lung tissue using single-cell RNA-seq datasets. The monocyte fraction was reduced upon VT107 treatment while myeloid cells' fraction that includes tissue resident macrophages was expanded along with a slight increase in leukocytes (FIG. 9, Panel I). Together this data demonstrates a TEAD dependent effect on the inflammatory profile in the lung microenvironment in both tumor naïve and tumor bearing mice.

### TEAD Inhibition Reprograms Lung Resident Macrophages and Enhanced IL12 Production

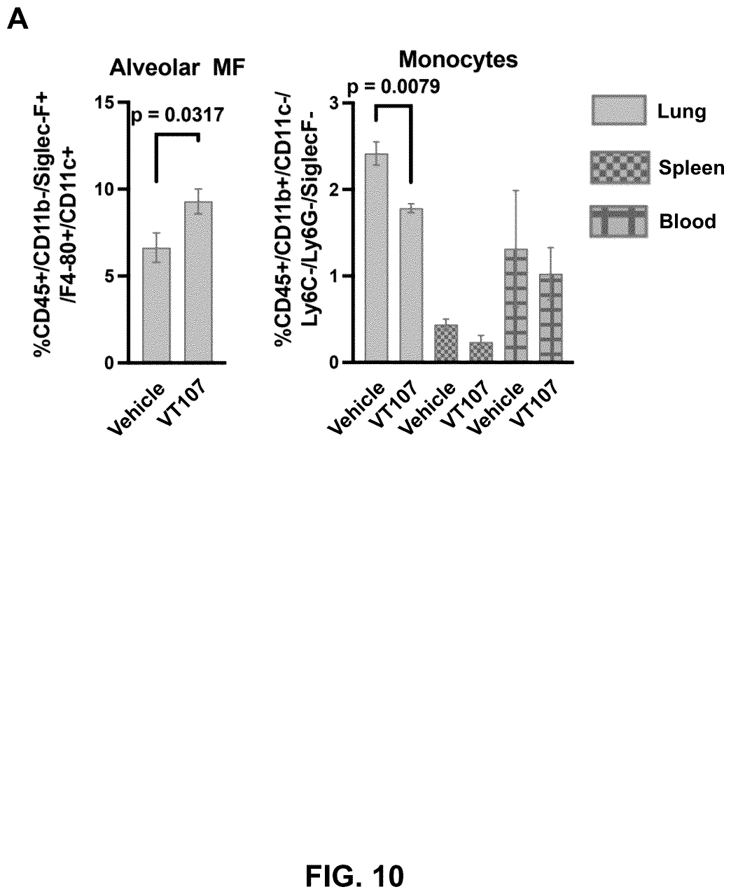

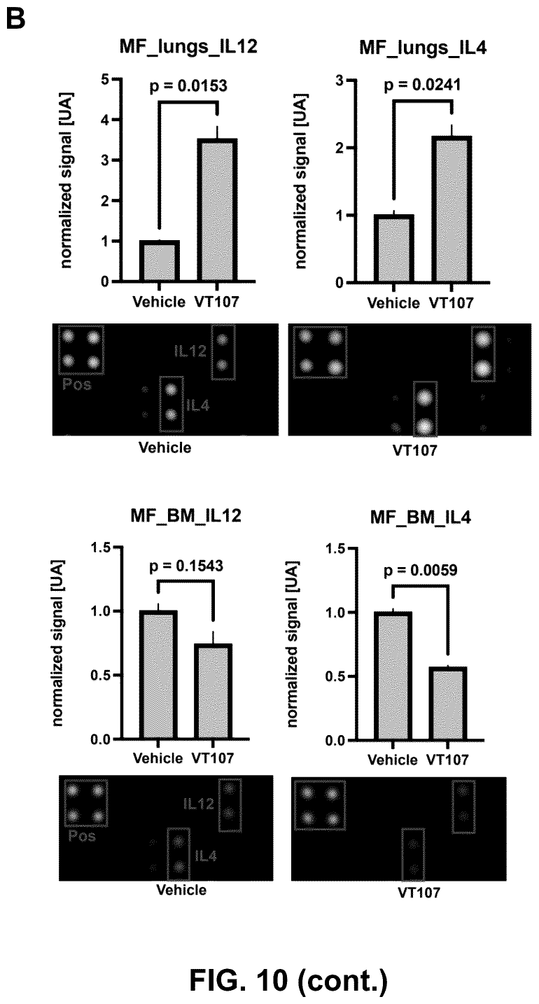

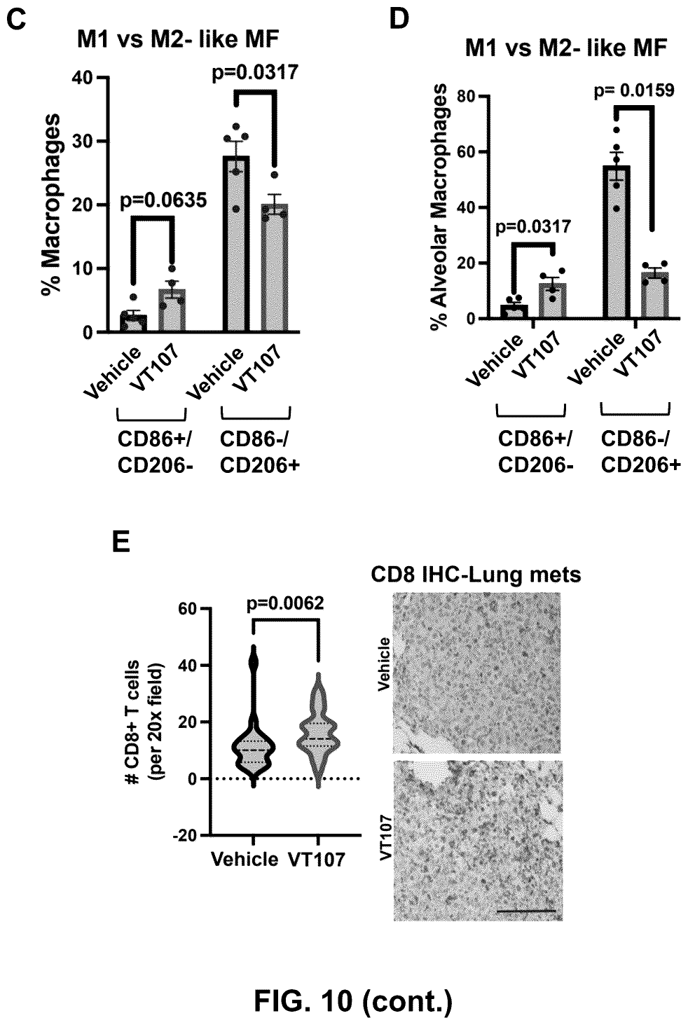

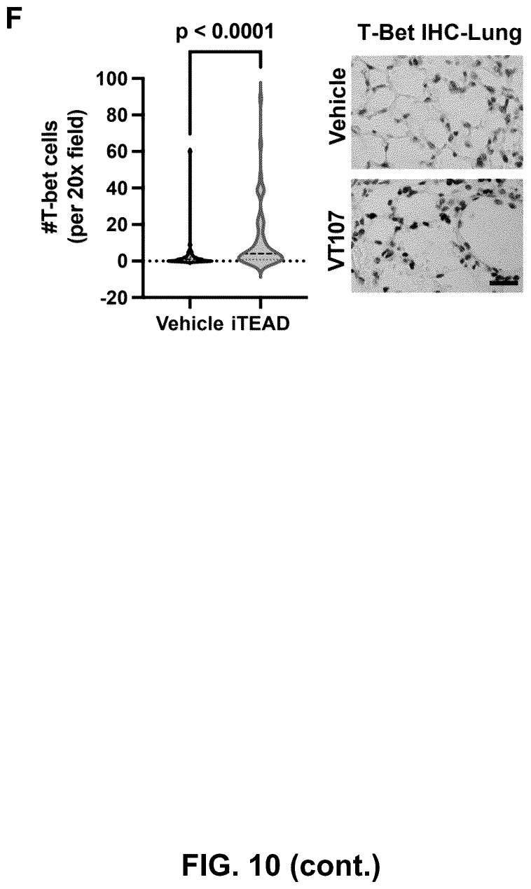

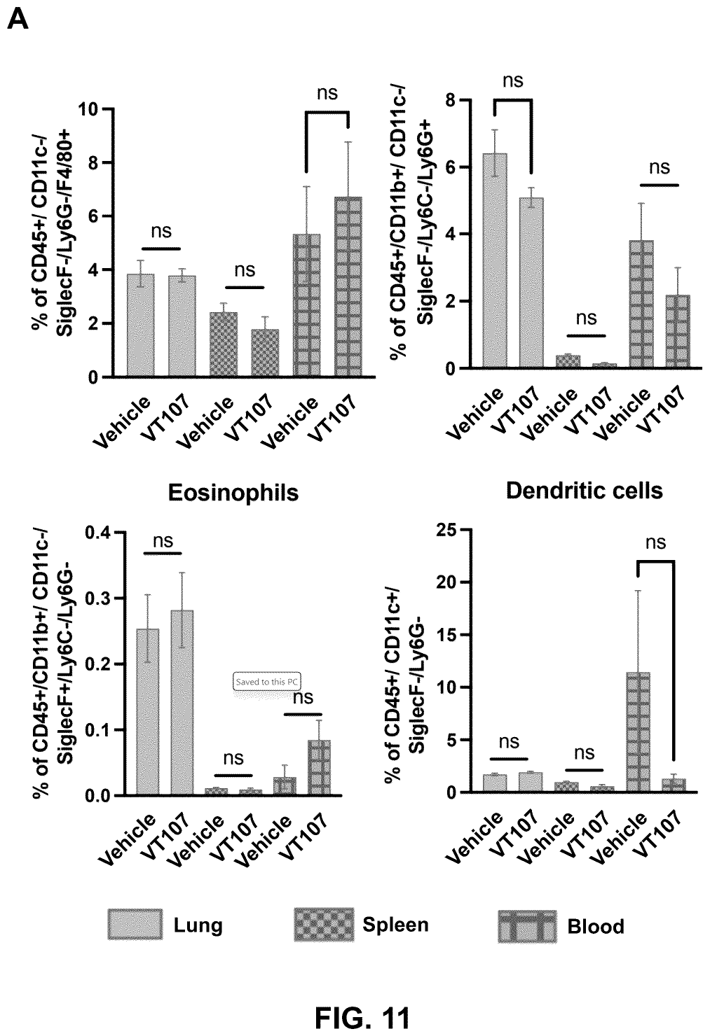

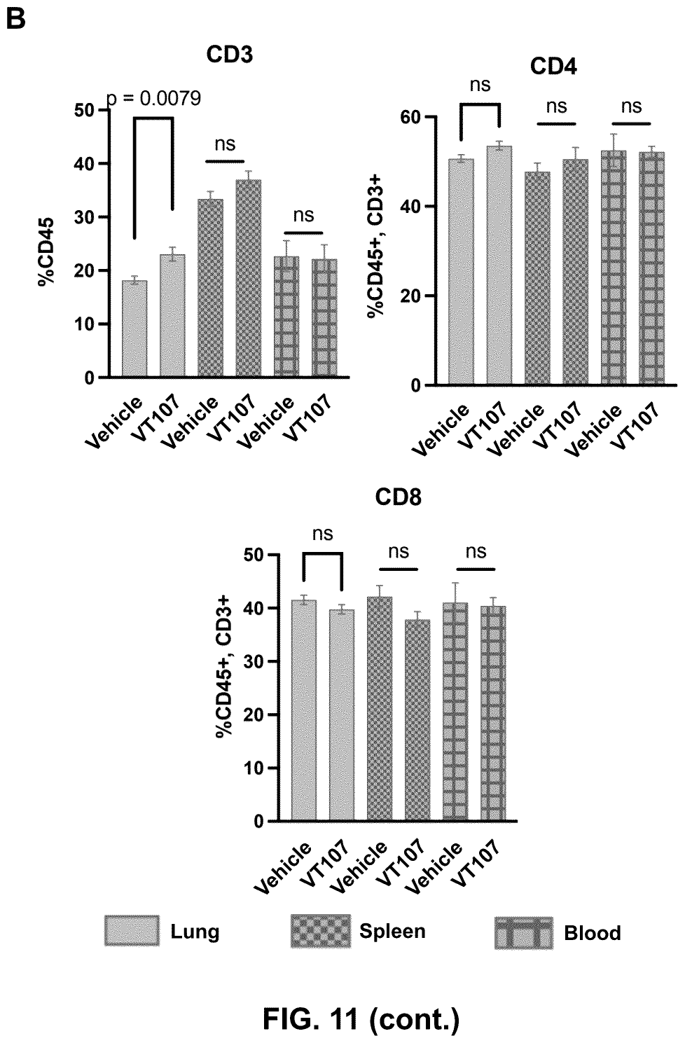

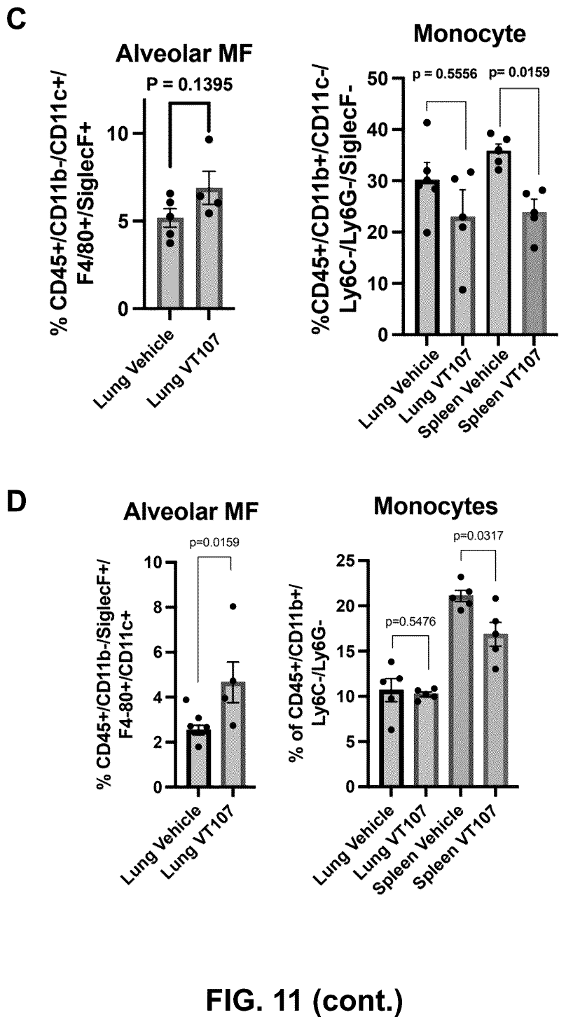

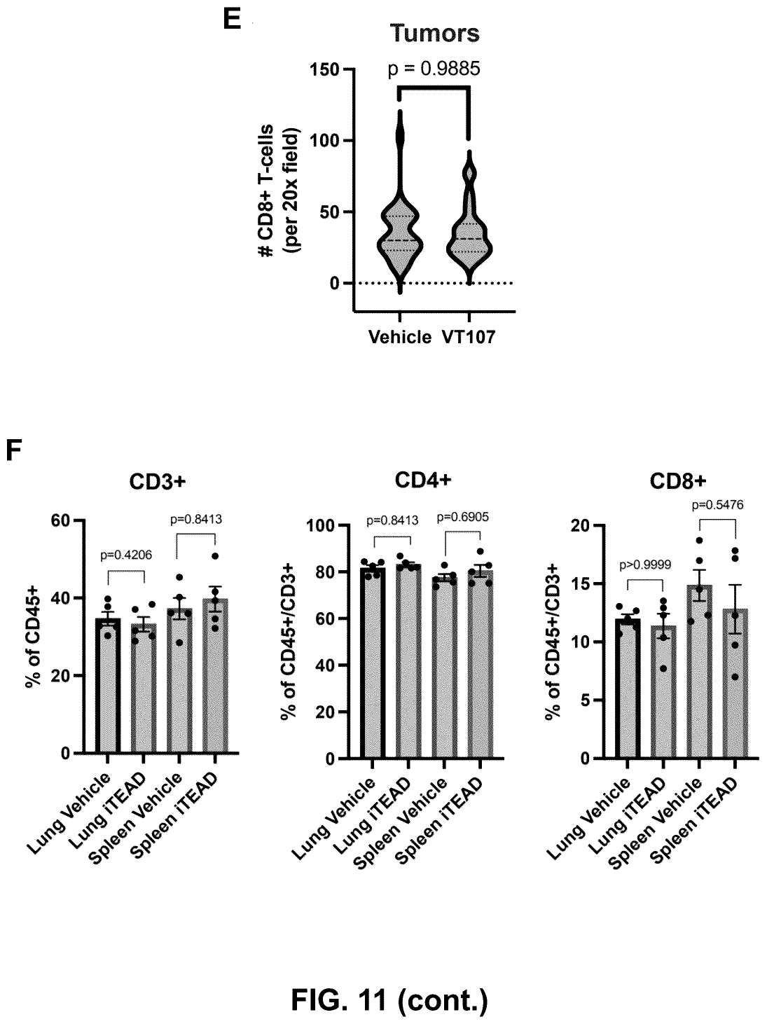

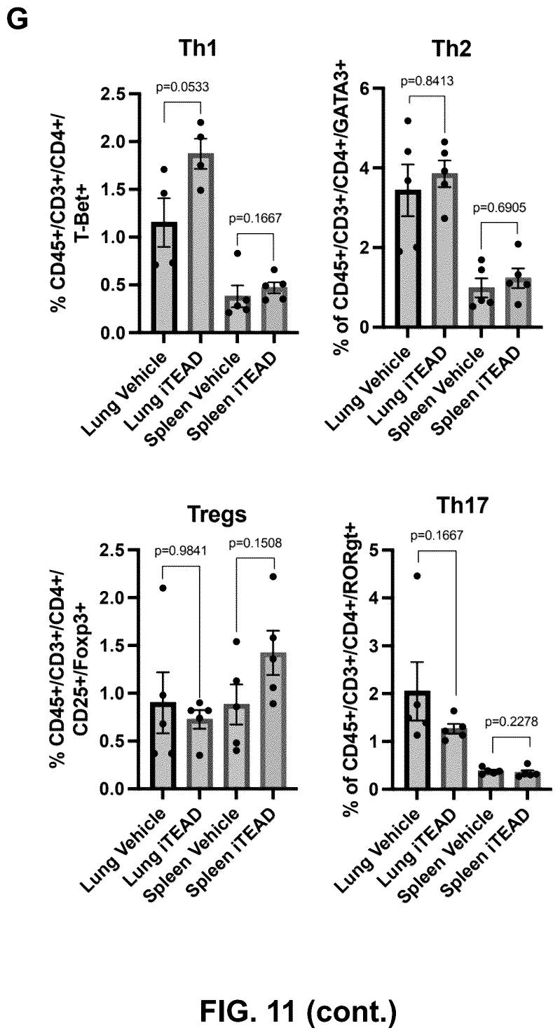

Due to the shifts in immune signaling observed in the lung tissue upon TEAD inhibition (FIG. 9), immune cell populations were first analyzed by flow cytometry in the lungs and spleens of tumor naïve mice. Consistent with the Cibersort analysis of RNA-seq data, there was a decrease in the monocytes population and an increase in the tissue-resident alveolar macrophages (AM) (FIG. 10, Panel A). No significant change in total macrophages, dendritic cells, eosinophils or neutrophils was observed in analyzed tissues (FIG. 11, Panel A). Flow cytometry analysis of the lymphoid cell population in the tumor naïve lungs showed an increase in CD3+ cells, but not specifically in the CD4+ or CD8+ T-cell populations (Fig S2B). These analyses suggest that VT107 treatment expanded the T-cell population in the lungs, but in the absence of tumor cells, no specific clonal expansion occurred. Next, it was sought to determine the specific effects of iTEAD treatment on lung resident versus bone-marrow derived macrophages, as the dynamics of these cell populations are crucial for the establishment of the metastatic niche (Ries et al., 2014; Qian et al., 2011). Macrophages were retrieved from the lungs by isolating F4/80+ cells, a specific marker for murine macrophages, by FACS (fluorescence-activated cell sorting) as well as bone marrow-derived macrophages from the femurs of naïve C57BL/6 mice that were then differentiated in vitro using macrophage colony stimulating factor (M-CSF). Macrophages were treated with 1 μM of VT107 for 24 hours before the conditioned media was collected and analyzed on a cytokine array. Lung resident macrophages secreted a significantly higher level of IL12 and IL4 upon VT107 treatment, whereas iTEAD had no effect on IL12 and reduced IL4 levels in bone marrow-derived macrophages. There was no change in the level of the other cytokines detected (FIG. 10, Panel B). This data further illustrates the differential effect of TEAD inhibition on lung resident versus infiltrating monocyte/macrophage populations.

Similar to tumor naïve lungs, tumor bearing lungs had a higher frequency of alveolar macrophages and a decrease in monocyte populations in iTEAD treated groups (FIG. 11, Panel C and D). Importantly, CD86, a marker of M1-like macrophages, was significantly enriched in the lung macrophage population while CD206, a marker of M2-like macrophages, was significantly reduced (FIG. 10, Panel C). This shift was also significant in the alveolar macrophage population with iTEAD treatment (FIG. 10, Panel D). Additionally, there was an increase in the number of CD8+ T-cells infiltrating within the lungs, but not the primary tumors (FIG. 10, Panel E and FIG. 11, Panel E). Notably, CD8+ T-cells were observed in the metastatic lesions of the VT107-treated group whereas a minimal number of CD8+ T-cells were observed in the metastases from the control group (FIG. 10, Panel E). To determine early changes in the T-cell population, lung and spleen tissues of BALB/c mice that were intravenously injected with 4T1 were processed and analyzed by flow cytometry 10 days post inoculation (FIG. 9, Panel E). There was no difference in the overall abundance of CD4+ and CD8+ T-cell populations at this time point between treatment groups (FIG. 11, Panel F), however, the helper 1 T-cell (Th1) population was increased in VT107-treated mice, but not other CD4+ T-cell subtypes (FIG. 11, Panel G). A significant increase in T-bet staining, a marker of Th1 CD4+ cells (Szabo et al., 2000), confirmed an enhanced Th1 cells' presence in the lungs of iTEAD-treated mice (FIG. 10, Panel F). iTEAD treatment reduced lung metastasis and shifted resident macrophages to an anti-tumor phenotype as well as increased T-cell subpopulations in the lung. M1-like macrophages and IL12 signaling play a critical role in the crosstalk between macrophages and T-cells triggering their proliferation and differentiation of T-cell subsets that can eliminate tumor cells unlike the pro-tumor M2-like macrophages (Guerriero, 2019; Oishi et al., 2016; Shao et al., 2023). Thus, the interplay between macrophage and T-cells after iTEAD treatment is likely to play a role in the increase in CD8+ T-cell and Th1 CD4+ cell populations in the lungs.

TEAD Knockdown Enhances Macrophage Crosstalk with Cancer Cells and Halts Metastatic Seeds' Outgrowth in the Lung of NOD/SCID Mice

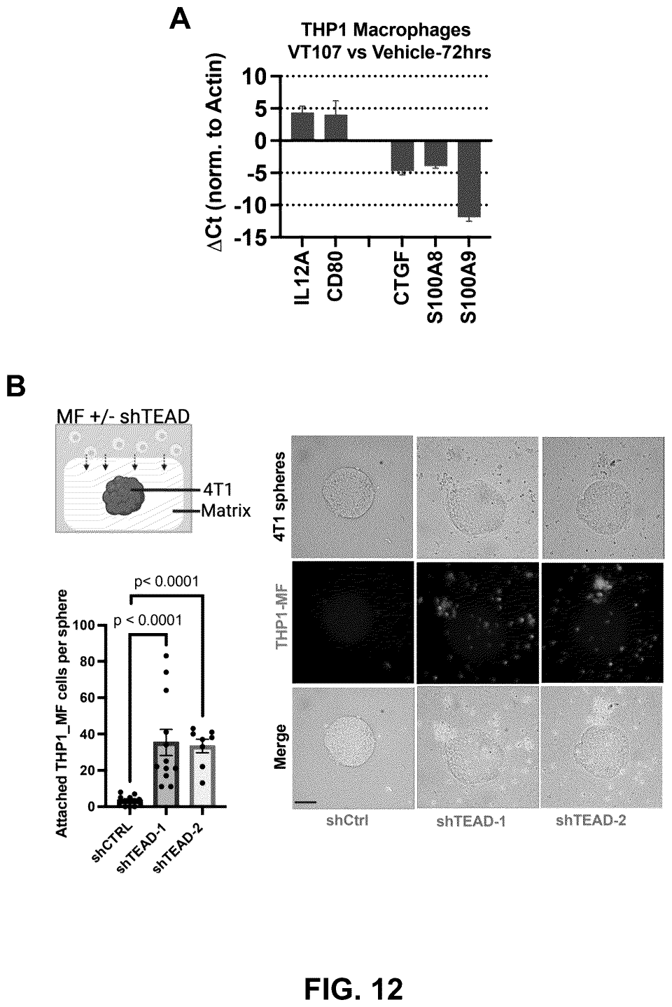

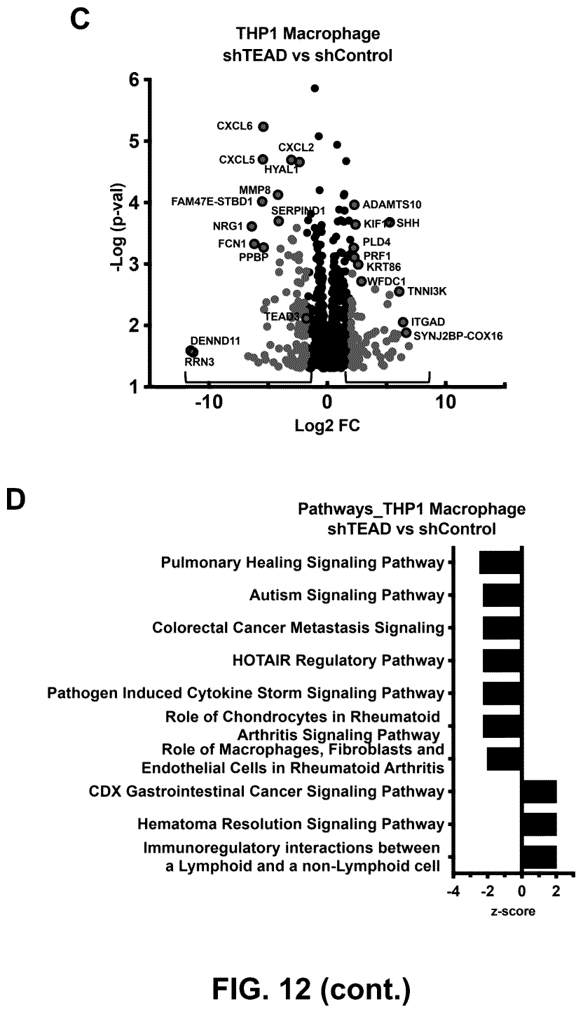

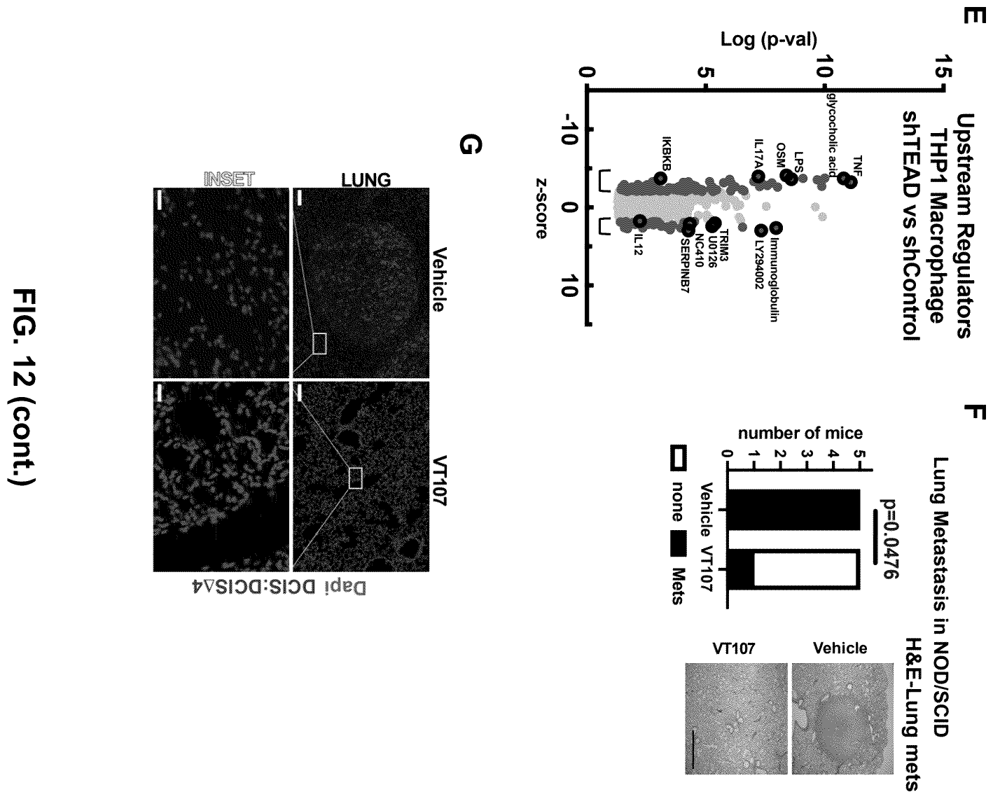

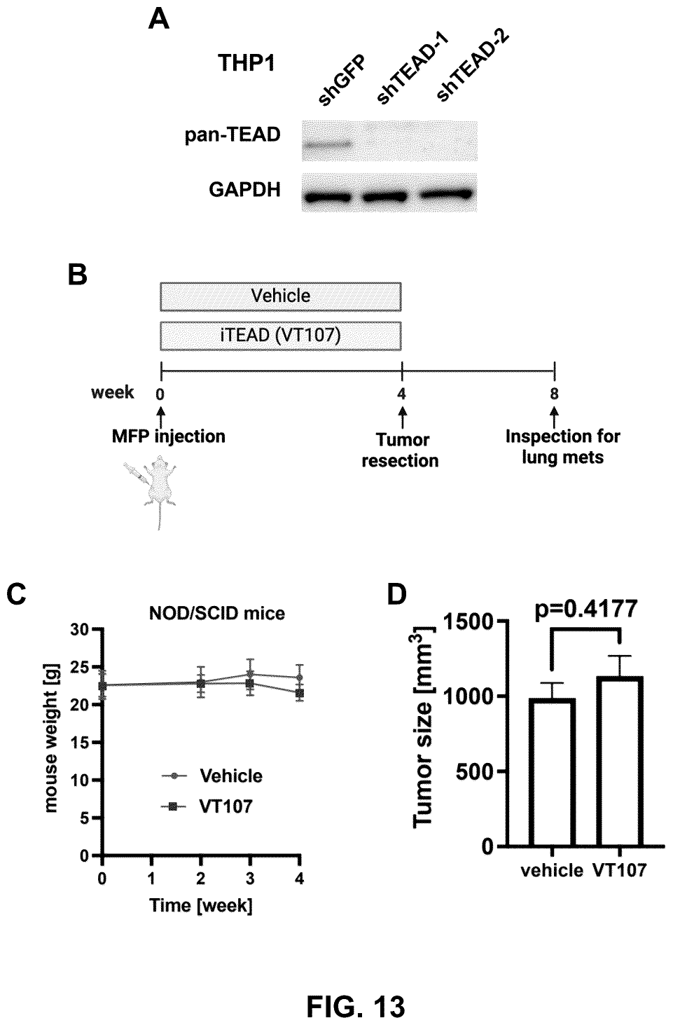

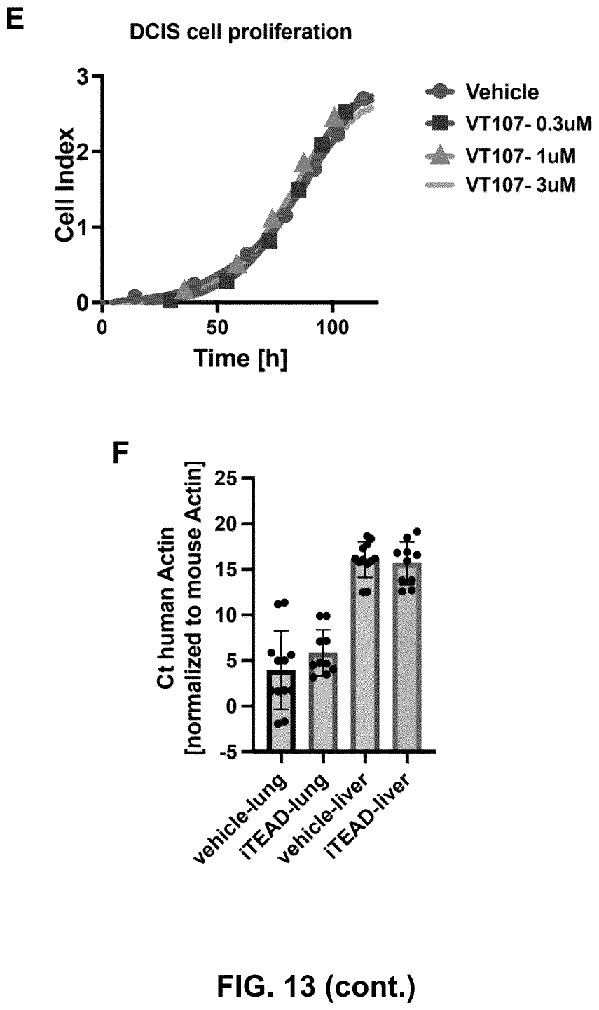

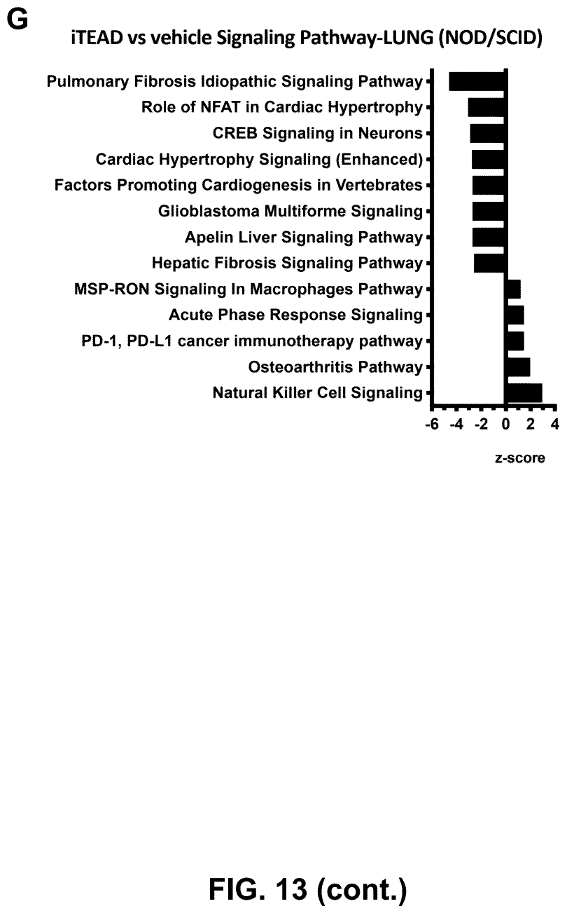

To further study the direct effects of TEAD inhibitors on macrophages, THP1, a human monocytic leukemia cell line that can differentiate into macrophages upon phorbol 12-myristate-13-acetate (PMA) treatment (Tsuchiya et al., 1980), was used. iTEAD treatment on these macrophages showed an increase in IL12 and CD80 gene expression levels and a decreased expression of the known YAP/TEAD target genes CTGF, S100Δ8 and S100Δ9 (FIG. 12, Panel A). To determine if knockdown of TEAD mimicked iTEAD effects, THP1 cells were transduced with shRNAs targeting TEAD 1, 3 and 4 or GFP as a control. Knockdown was confirmed by western blot with a pan TEAD antibody (FIG. 13, Panel A). TEAD knockdown THP-1 macrophages migrated and attached to tumorspheres embedded in matrix compared to no effect in control macrophages (FIG. 12, Panel B). TEAD knockdown in THP1 macrophages decreased protumor inflammatory cytokines CXCL2, 5, and 6 (FIG. 12, Panel C). The top upregulated pathway was related to the immune interactions between lymphoid and non-lymphoid cells, a process necessary to mount an immune response (FIG. 12, Panel D) and is consistent with the enhanced crosstalk we observed between tumorspheres and shTEAD THP1 macrophages (FIG. 12, Panel B). Signaling pathways related to inflammatory diseases, such as rheumatoid arthritis, were decreased (FIG. 12, Panel D), as well as the inflammatory upstream regulators LPS and TNF (FIG. 12, Panel E). The score for M2-like upstream regulators, OSM (Shrivastava et al., 2019) and IL17A (Miller et al., 2020) was decreased by TEAD knockdown yet importantly, IL12 and the immune modulator used to enhance immunotherapy, NC410, scores were increased (Ramos et al., 2021) (FIG. 12, Panel D). Taken together, the response to iTEAD treatment or TEAD knockdown enhanced macrophages interaction with tumor cells and IL12 signaling. The global anti-inflammatory effect and the repression of the M2-like macrophage phenotypes are known to oppose the establishment of a hospitable metastatic niche (McGinnis et al., 2024; Sica et al., 2008; Noy & Pollard, 2014).

To further examine the effect of TEAD inhibition on the myeloid compartment of the lung, a human breast cancer cell line MCFDCIS (DCIS) that metastasizes to the lungs when a small population of cells (DCISA4) expresses an isoform of the transcription coactivator amplified in breast cancer 1 (Sharif et al., 2021), was used. DCIS:DCISA4 cells were injected into the mammary fat pad of NOD/SCID mice. Mice were treated daily with VT107. Tumors were surgically removed at week 4 and lungs were monitored for metastases for another 4 weeks without receiving additional treatment (FIG. 13, Panel B). Mouse body weights were measured weekly throughout the course of treatment (FIG. 13, Panel C). Primary tumor size was similar between vehicle and VT107 treated mice (FIG. 13, Panel D), which was consistent with in vitro data showing no significant change in DCIS proliferation in the presence of VT107 at varying concentrations (FIG. 13, Panel E). All mice in the vehicle treated group developed lung metastases as previously reported (Sharif et al., 2021). In contrast, the VT107-treated group showed no overt metastases in 4 out of 5 mice (FIG. 12, Panel F). However, detection of human actin in lung tissues by qPCR, relative to mouse actin levels, indicated the presence of DCIS cells in the lungs of VT107-treated mice (FIG. 13, Panel F). Immunofluorescent staining of the DCIS:DCISA4 cells confirmed the presence of single cancer cells in the lungs that failed to grow into overt metastases in the VT107-treated group (FIG. 12, Panel G). This data suggests that TEAD inhibitors could directly influence the microenvironment to hinder the metastatic outgrowth of cancer cells that have already seeded the lungs. RNA was extracted from lung tissues to further examine the host stroma affected by VT107. RNA sequencing reads from the lungs were aligned to the mouse transcriptome to shed light on the host tissue changes in response to treatment in the presence of seeded cancer cells. Similar to the findings in immunocompetent mice, pathway analysis of gene expression changes revealed an upregulation of myeloid cell activating pathways, acute phase response signaling and PD-L1 immunotherapy pathways. Signaling pathways implicated in pulmonary and hepatic fibrosis, cardiac hypertrophy and liver injury were downregulated (FIG. 13, Panel G). These observations again show a shift in the inflammatory phenotypes in the microenvironment which could be responsible for the halt in the metastatic outgrowth of seeded cancer cells in the treated group.

### TEAD Inhibition Enhances Early Macrophage-T-Cell Crosstalk Via IL12

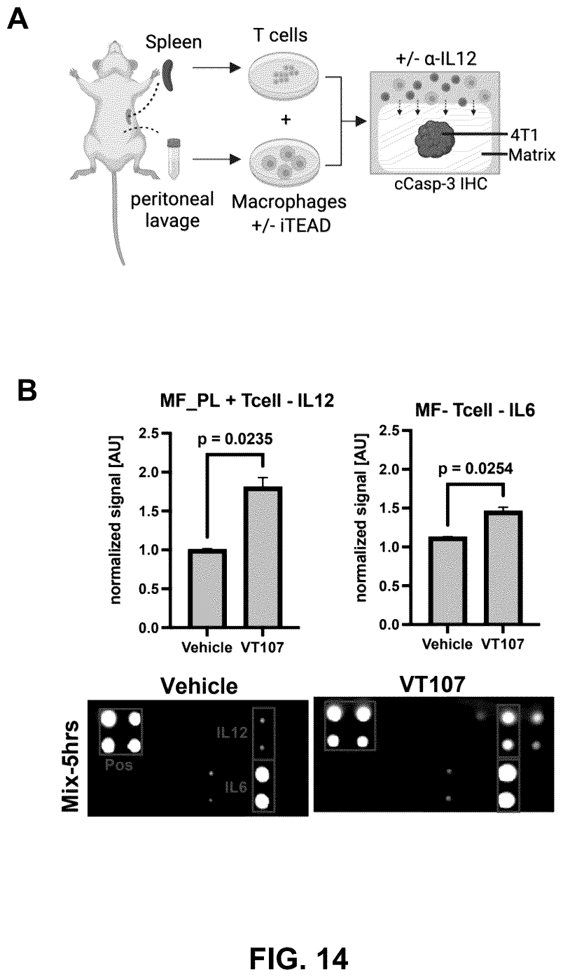

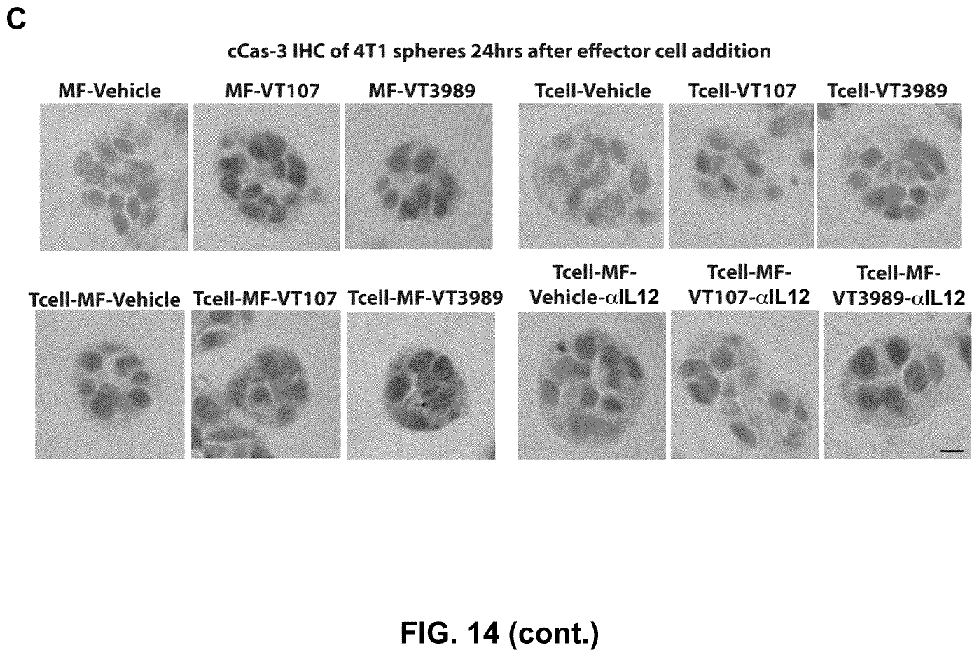

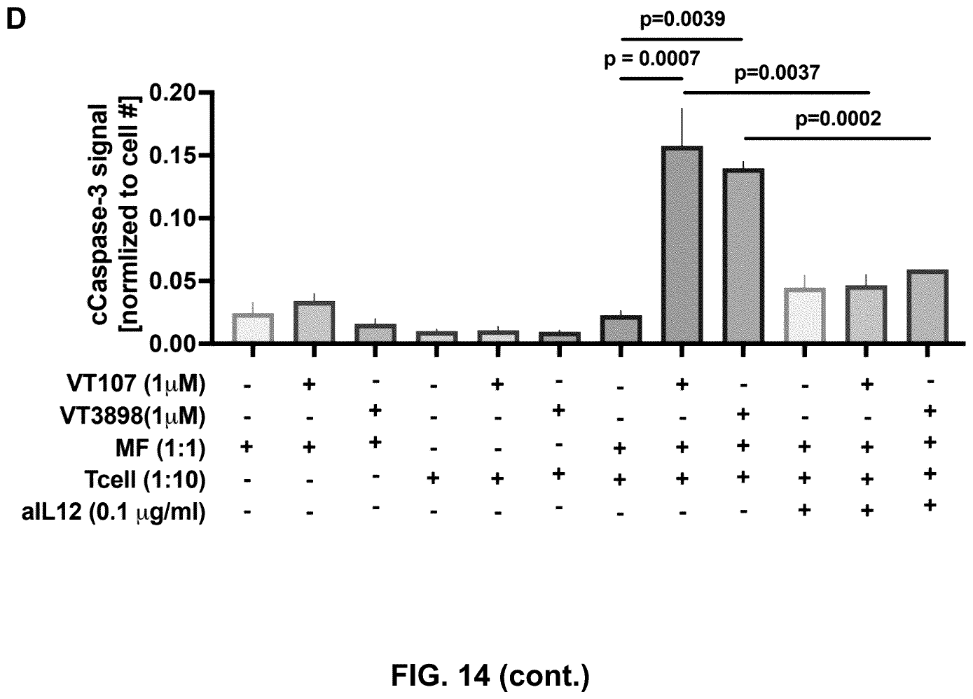

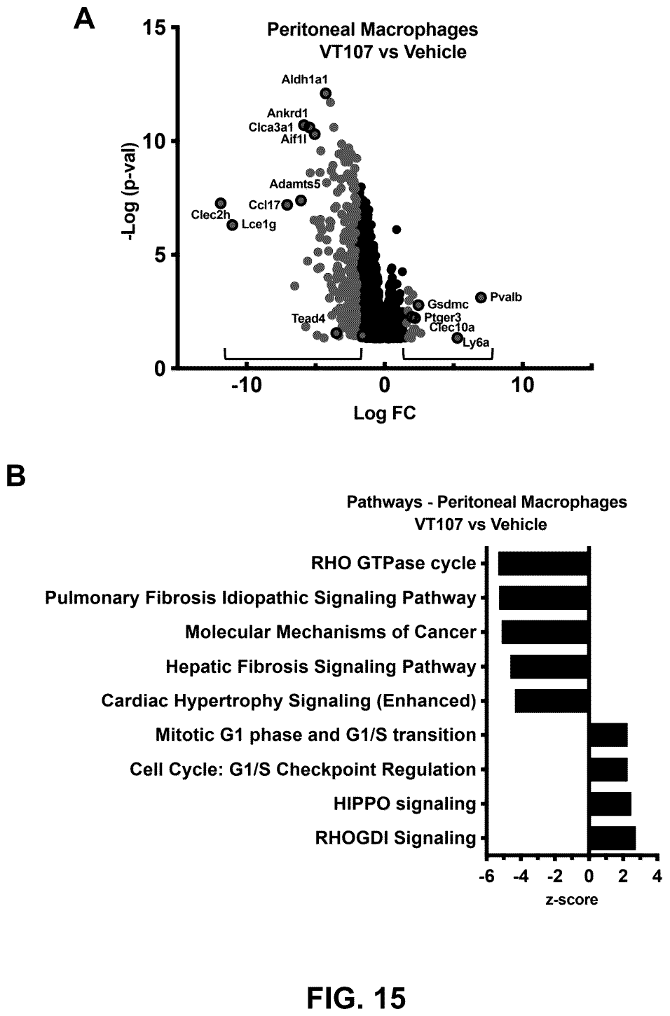

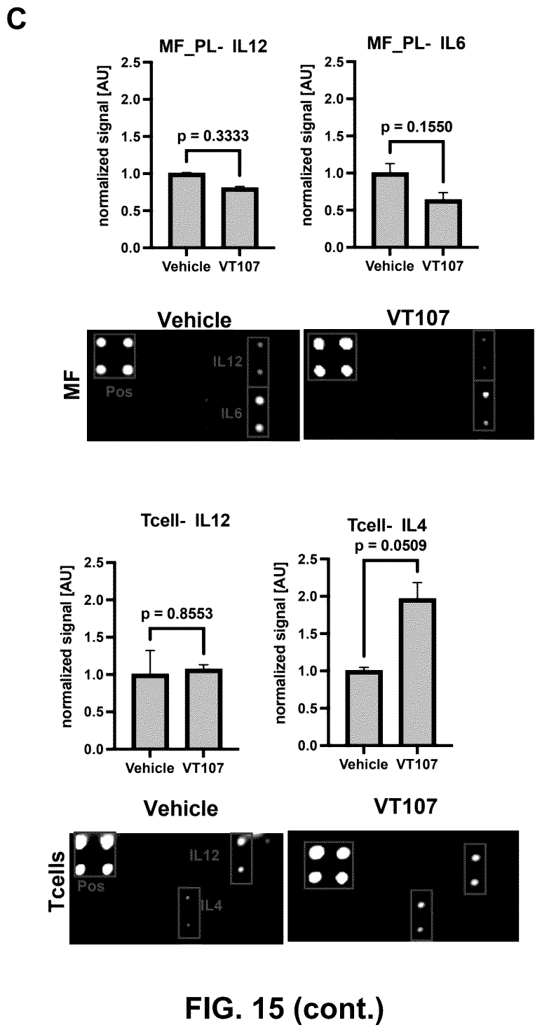

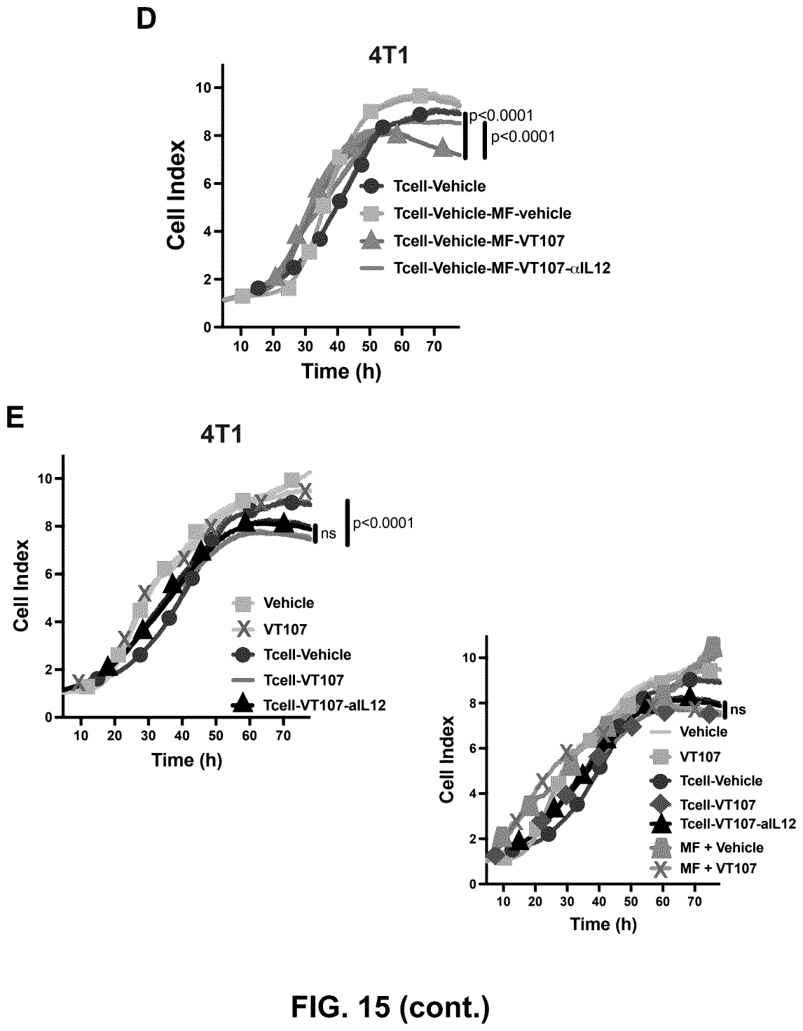

To investigate whether the effect of iTEAD treatment on macrophages can enhance their crosstalk with T-cells in the lung microenvironment, peritoneal macrophages+/−iTEAD were first isolated and characterized as a model of resident macrophages for ex vivo experiments (FIG. 14, Panel A). It was observed that treatment with VT107 decreased gene expression of M2-like and fibrosis regulators, Aldh1a1 and Cc117 (Chen et al., 2020), as well as TEAD4 and its downstream target gene Ankrd1. Pvalb, an inhibitor of M2-like phenotype, was the top upregulated gene (Lin et al., 2022) (FIG. 15, Panel A). Pathway analysis showed downregulation of disease-related chronic inflammation while cell cycle related pathways were upregulated (FIG. 15, Panel B). The iTEAD pretreated peritoneal macrophages and untreated T-cells isolated from the spleen of tumor naïve mice (FIG. 14, Panel A) were then mixed for 5 hours before collecting the conditioned media for analysis. IL12 and IL6 levels were significantly increased in the cell mix when macrophages were pretreated with iTEAD inhibitor (FIG. 14, Panel B). No significant change was observed in IL12 or IL6 levels in the iTEAD-treated macrophages or T-cells alone (FIG. 15, Panel C). IL4 was slightly increased in T-cells after treatment (FIG. 15, Panel C). Next, these immune cell mixes were added to 4T1 spheres that were embedded into matrix. An elevated signal of cleaved caspase-3 in 4T1 cells, a marker of cellular apoptosis, was observed in the presence of iTEAD-treated macrophages and T-cells after 24 hours (FIG. 14, Panels C and D). This effect was reversed by an IL12 neutralizing antibody. No change in cleaved caspase-3 in 4T1 cells in the presence of VT107-treated T-cells or macrophages alone, was detected at 24 hours (FIG. 14, Panels C and D). 4T1 cells were then plated and allowed to attach on electrode-coated arrays to monitor cancer cell viability before the addition of macrophages+/−iTEAD and T-cells. VT107-treated macrophages significantly increased T-cells' cytotoxicity compared to T-cells alone and this crosstalk was diminished by neutralizing antibodies for IL12 (FIG. 15, Panel D). This reversal of T-cell cytotoxicity by IL12 antibodies was not observed in VT107-treated T-cells in the absence of macrophages (FIG. 15, Panel E) indicating a critical role for IL12 in the crosstalk between macrophages and T-cells.

### TEAD Inhibition Activates T-Cells and Enhances Degranulation

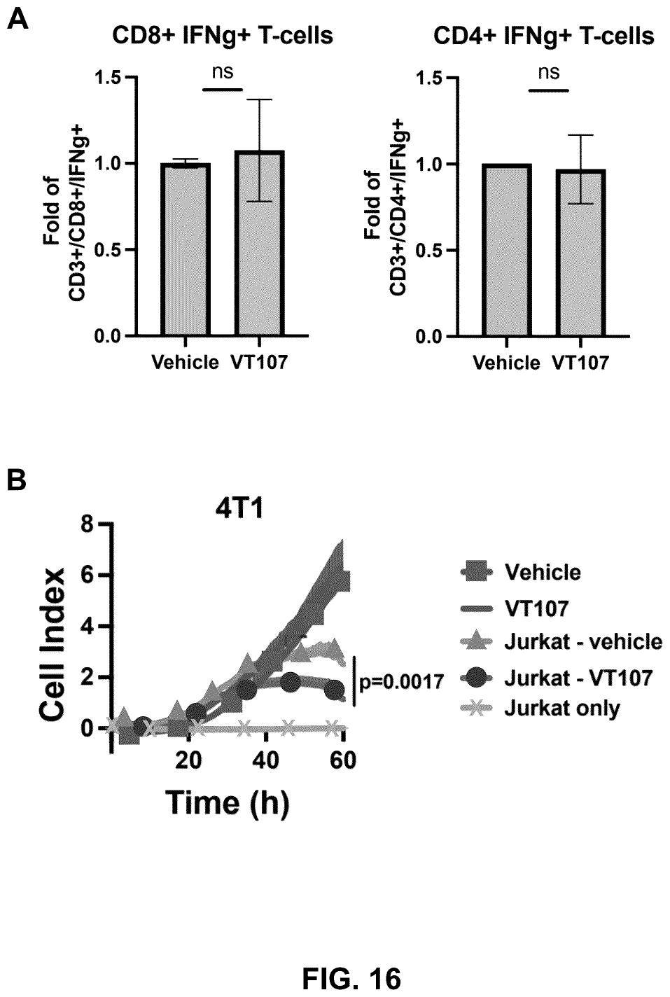

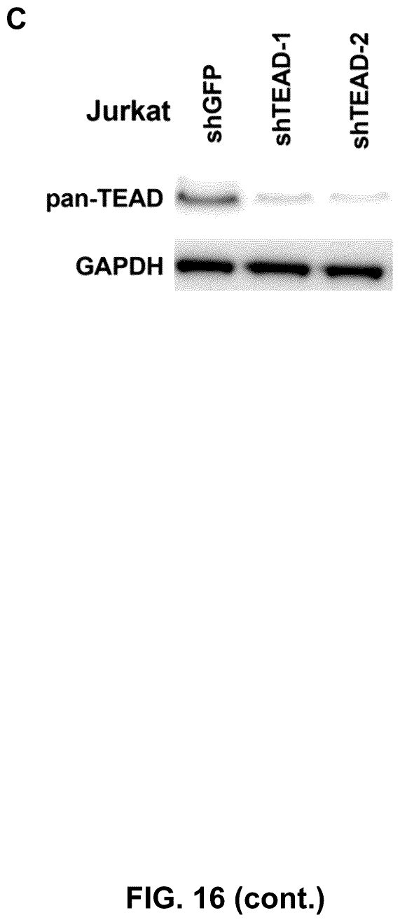

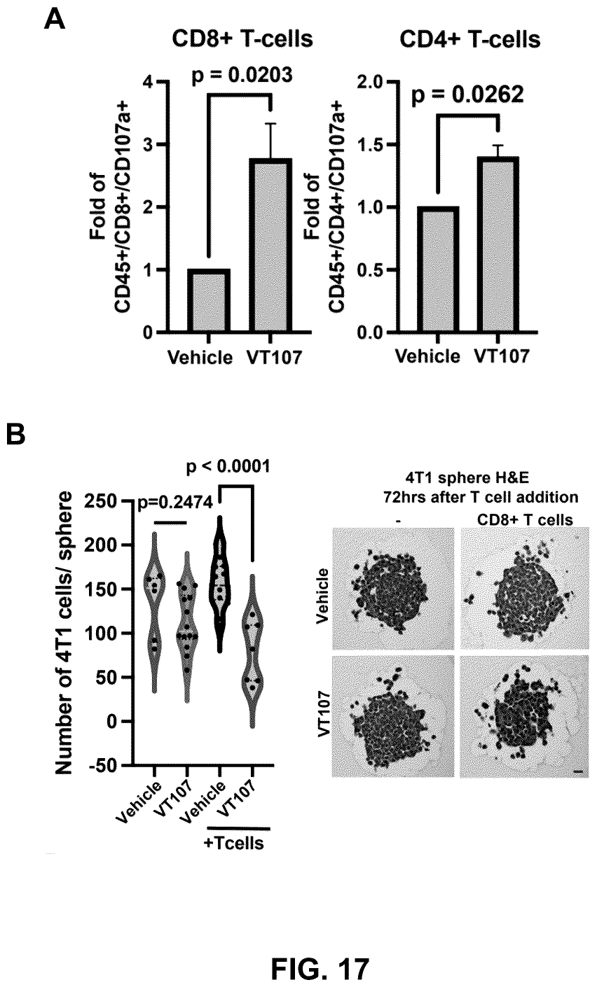

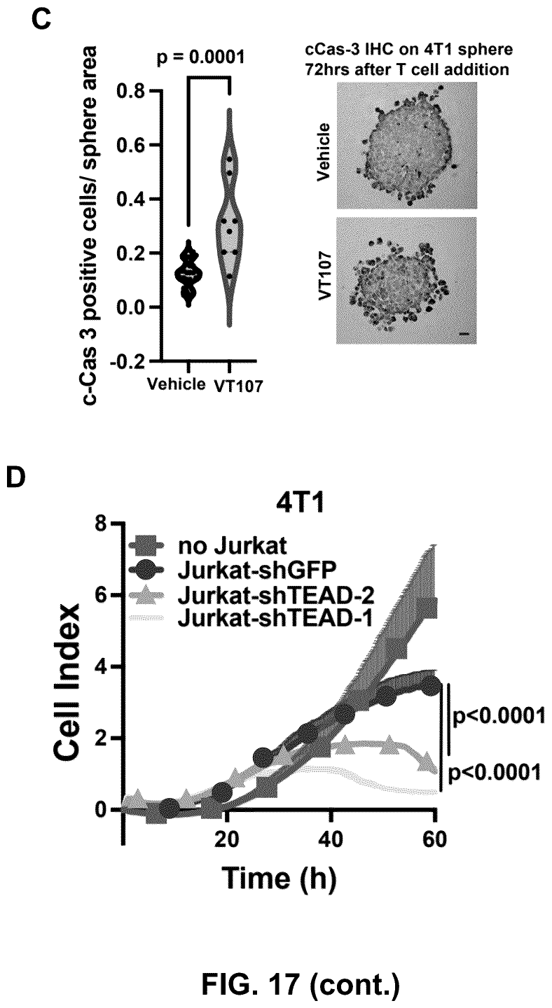

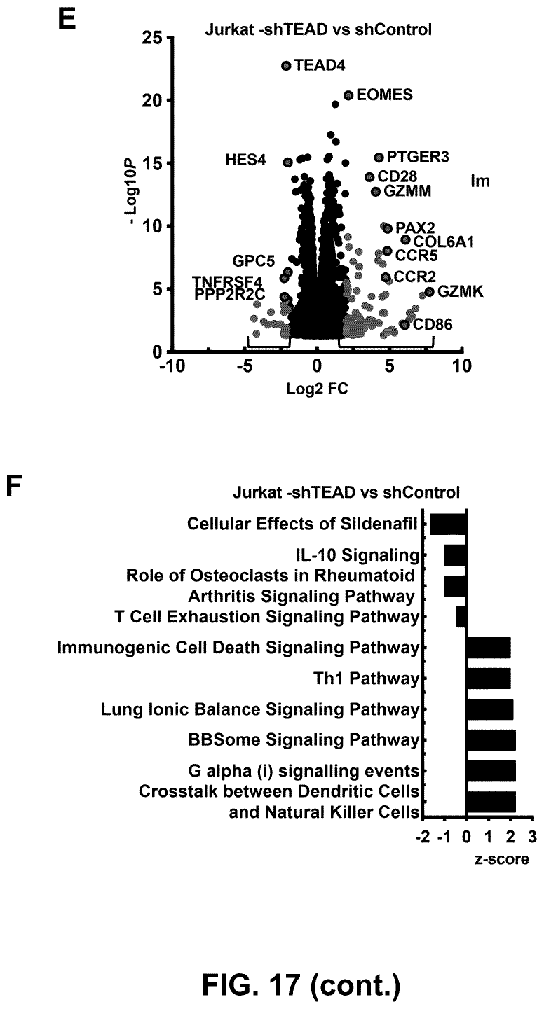

The hippo pathway has been implicated in T-cell development and function (Du et al., 2014; Ueda et al., 2012). For example, YAP expression is essential for tumor immune suppression by Treg cells that attenuates CD8 T-cell immunity (Geng et al., 2017; Ni et al., 2018; Lebid et al., 2020). Extended VT107 treatment of T-cells alone resulted in a slight increase in cytotoxicity as observed in FIG. 15, Panel D but not in short term treatment as observed in FIG. 14, Panel C. To investigate whether VT107 directly affected T-cells, T-cells treated with VT107 were co-cultured with 4T1 cells in 2D for 72 hours. There was no change in IFNγ levels in the conditioned media (FIG. 16, Panel A), however, the T-cell degranulation marker CD107a, was significantly increased which is indicative of cytotoxicity (Peters et al., 1991) (FIG. 17, Panel A). In 3D, the number of 4T1 cells per sphere was significantly reduced when co-cultured with T-cells that were treated with VT107 for 72 hours compared to vehicle (FIG. 17, Panel B). Cleaved caspase-3 immunohistochemistry (IHC) staining was also increased with treatment (FIG. 17, Panel C). To further confirm T-cell activation upon TEAD activity reduction, we used Jurkat cells, a CD4 positive human lymphoma cell line (Schneider et al., 1977). After treatment with 1 μM of VT107, Jurkat cells showed enhanced cytotoxicity towards co-cultured mouse 4T1 cells (FIG. 16, Panel B). Jurkat cells' cytotoxicity was also significantly increased in shTEAD-transduced lines, targeted at TEAD 1,3 & 4 (FIG. 16, Panel C), compared to control (FIG. 17, Panel D). RNA-seq analysis of these TEAD knockdown Jurkat lines revealed a significant upregulation of the T-cell survival and activation markers CD28 and CD86 (Linsley & Ledbetter, 1993; Paine et al., 2012) and cytotoxicity granzymes K and M as well as Th1 cell differentiation markers CCR5 and EOMES (Contento et al., 2008; Thelen et al., 2023) (FIG. 17, Panel E). Signaling pathway analysis showed increase in immune activation and downregulation of T-cell exhaustion signaling pathways and IL10 signaling (Oft, 2014) (FIG. 17, Panel F). This data is consistent with previous reports of YAP/TEAD signaling impacting T-cell activation. However, here we show the specific effect of TEAD reduction on granzyme gene expression and degranulation that result in the killing of cancer cells.

### Methods

### Real Time Cell Analysis

Cell analysis was done using E-plates from xCELLigence, (Agilent, #5469830001) according to the manufacturer's protocol. For cell cytotoxicity, cancer cells (10,000-25,000 cells per well) were seeded and allowed to attach for 1-2 hours prior to the addition of immune cells and/or drug treatments. For cell proliferation, cancer cells were seeded at 5,000-10,000 cells per well with incremental doses of drug treatments or DMSO as a vehicle control. Cell index was measured by changes in electric impedance at 15 min intervals. TEAD inhibitors were obtained from Vivace therapeutics.

### Histology

Formalin-fixed, paraffin embedded (FFPE) lung tissue sections were stained with hematoxylin and eosin. Immunohistochemistry staining was performed on FFPE tumor and lung tissue sections. Slides were deparaffinized in xylene and rehydrated following a series of ethanol washes. Heat-induced antigen retrieval was then performed by placing slides in warmed sodium citrate buffer (pH 6.0) (Invitrogen, 005000) in a pre-heated steamer for 20 minutes. Slides were cooled at room temperature for an additional 20 minutes, prior to quenching endogenous peroxidase activity using 3% hydrogen peroxide buffer (Fisher, BP2633500) for 10 minutes. Next, slides were incubated overnight at 4° C. with either anti-CD8 (Cell Signaling Technology, #98941), anti-CD68 (Abcam, ab303565), anti-CD206 (Cell Signaling Technology, #24595), or anti-F480 (Cell Signaling Technology, #70076). Following PBS washes, the slides are then stained with a biotinylated secondary antibody using the VECTASTAIN Elite ABC-HRP Kit according to the user's manual (Vector Laboratories, PK-6101). Positive brown staining was produced by applying ImmPACT DAB EqV Substrate Kit (Vector Laboratories, SK-4103) to the slides, which were then counterstained with hematoxylin (Sigma, MHS16). Images were captured using the Olympus BX40 microscope, and positive staining was quantified manually. Immunofluorescent staining was performed on lung tissue sections. Slides were baked at 60°, deparaffinized in xylene, rehydrated, washed in DI water and incubated with 10% neutral buffered formalin (NBF) for an additional 20 minutes to increase tissue-slide retention. Epitope retrieval/microwave treatment (MWT) for all antibodies was performed by boiling slides in Antigen Retrieval buffer 6 (AR6 pH6, Akoya AR6001KT). Protein blocking is performed using antibody diluent/blocking buffer (Akoya, ARD1001EA) for 10 minutes at room temperature. Slides were incubated with Luciferase antibody (Sigma, #L0159) for 1 hour and OPAL Fluoresce 620 then counterstained with spectral DAPI (Akoya FP1490) for 5 min and mounted with ProLong Diamond Antifade (ThernoFisher, P36961). Slides were scanned at 10× magnification using the Vectra 3.0 Automated Quantitative Pathology Imaging System (PerkinElmer/Akoya).

### Western Blot

Cells were lysed in NP40 lysis buffer, in the presence of cOmplete Protease Inhibitor Cocktail (Roche, #12352204), and a phosphatase inhibitor Na3VO4. Lysates were separated on an SDS-PAGE gel and immunoblotted with antibodies against pan-TEAD (Cell Signaling Technology, #13295), and GAPDH (Cell Signaling Technology, #2118).

T-Cell Isolation from Tissues

Mouse lungs and tumors were digested using the Tumor Dissociation Kit (Miltenyi Biotec, #130-095-929) as described in the manufacturer's protocol. Tumors and lungs were subdued to mechanical dissociation for 60 and 30 minutes, respectively. Spleens were passed through a 100 um cell strainer (Fisherbrand, #22-363-549) using a syringe plunger. T-cells were isolated from all dissociated tissues using the EasySep Mouse T-Cell Isolation Kit (StemCell Technologies, #19851).

### Bone Marrow Isolation and Macrophage Differentiation

The bone marrow was flushed with 100 ul phosphate buffered saline (PBS) from the femur and tibia of a BALB/c mouse, treated with ACK lysis buffer (Thermofisher, Δ1049201) and cultured in RPMI medium with 10% FBS. Cells were cultured with 10 ng/ml mouse M-CSF (StemCell, 78059.1) for 3 days then washed with PBS to get rid of floating cells. Adherent cells (macrophages) were treated with vehicle or 1 M VT107 for 24 hours.

### Flow Cytometry

Single cell suspensions were washed 2× with PBS. Cells were then stained with Zombie NIR Fixable Viability Kit (Biolegend, 423106) or Zombie Aqua Fixable Viability Kit (Biolegend, 423102) for 20 minutes at room temperature, to identify dead cells. Next, cells were washed with FACS buffer (1×PBS+2% FBS) and incubated with conjugated antibodies binding to cell surface proteins, at 4□C for 30 minutes. A combination of the following antibodies were used: anti-CD45-Spark Blue 550 (Biolegend, 103166), anti-CD3-PercP/Cy5.5 (Biolegend, 100217), anti-CD4-Pacific Blue (Biolegend, 116007), anti-CD8-BV785 (Biolegend, 100750), anti-CD25-BV650 (Biolegend, 102038), anti-CD45-AF700 (Biolegend, 103128), anti-CD45R (B220) (Biolegend, 563103), anti-SiglecF-APC (Miltenyi Biotec, 130-102-241), anti-CD11b-PE/Dazzle (Biolegend, 108745), anti-CD11c-FITC (Biolegend, 117306), anti-F480-PE/Cy7 (Biolegend, 123114), anti-Ly6G-BV785 (Biolegend, 127645), anti-Ly6C-BV570 (Biolegend, 128030), anti-CD206-AF700 (Biolegend, 141734), and anti-CD86-AF488 (Biolegend, 105018). Cells were fixed and permeabilized using the BD Cytofix/Cytoperm Fixation/Permeabilization Kit (BD Biosciences, #554714) prior to incubation with anti-Foxp3-AF647 (Biolegend, 126407), anti-Gata3-PE (Biolegend, 653803), anti-RORy t-BV421 (Biolegend, 562894), and anti-T-bet-BV711 (BD Biosciences, 4B10). After staining, cells were washed 2× and subjected to flow cytometry analysis using the BD FACSymphony Δ3 flow cytometer analyzer. Results were analyzed using FCS Express 7 Research (De Novo Software).

### Degranulation Assay of T-Cells

T-cells were isolated from the spleens of naïve BALB/c as described above and treated in vitro with DMSO control or VT107 for 24 hours. T-cells were then co-cultured with 4T1 cells with the same treatment conditions. After 72 hours, all cells were collected and subdued to flow cytometry using anti-CD45-AF700 (Biolegend, 103128), anti-CD8-BV785 (Biolegend, 100750), anti-CD4-Pacific Blue (Biolegend, 116007), anti-CD107a-PE/Cy7 (Biolegend, 121620), and Helix NP Blue (Biolegend, 425305). Analysis was performed on the BD FACSymphony Δ3 flow cytometer analyzer, and results were analyzed using FCS Express 7 Research (De Novo Software).

### Cytokine Array

Cells were grown in their recommended cell culture media and treated with 1PM VT107 for 24 or 72 hours. Conditioned media was collected and passed through a 0.2 micron PES filter then analyzed on a RayBio® Mouse Cytokine Array (#AAM-TCR-1) according to the manufacture's protocol. The array includes two spots for each cytokine. The luminescence signal was normalized to the positive control on each membrane, then to the vehicle treatment signal of each cytokine.

### Animal Experiments

Studies in mice were reviewed and approved by the Georgetown University Animal Care and Use Committee (GUACUC). Animals were randomized to receive vehicle control (DMSO) or VT107. Treatment was formulated in 5% DMSO, 10% solutol and 85% D5W; D5W. 5% glucose and administered daily by oral gavage. Five hundred thousand cells were injected into the mammary fat pad of age-matched 8 weeks old female NOD/SCID or C57/B16 mice purchased from Charles River or Jackson Laboratory, respectively. A hundred thousand 4T1 cells were injected into the tail veins of BALB/c mice purchased from Jackson Laboratory. Experimental timelines, treatment start times and durations are indicated in the figure diagrams and figure legends. Tissues collected were preserved in RNAlater (Thermofisher, #AM7020) or fixed in 10% formalin for histology analysis.

### RNA Sequencing

The total RNA from tissue or cell lines was extracted using RNeasy Mini Kit (Qiagen, #74104) according to the manufacturer's instructions. Total RNA integrity was assessed with the Agilent 2100 Bioanalyzer. The library preparation and next-generation sequencing (NGS) were performed at Novogene Corporation Inc. (Sacramento, CA, USA). At least triplicate samples per experimental condition were analyzed, raw sequence reads (150 bp paired end, >45×106 average reads/sample) were aligned to the human or mouse transcriptome using STAR, and aligned reads translated to expression counts via featurecounts, followed by a standard edgeR (RRID:SCR_012802) pipeline to identify DEGs under specific conditions. Ingenuity pathway analysis (IPA, RRID:SCR_008653) was undertaken using a list of all differentially expressed genes with a cutoff of p-value less than 0.05 to identify signaling pathways and upstream regulators.

Cell Lines and shRNA

MCFDCIS (RRID:CVCL_5552) cell line was maintained in DMEM/F12 (1:1) medium (Gibco, Waltham, MA, USA, 11039-021) with 5% horse serum, 20 μg/mL epidermal growth factor (EGF), 100 μg/mL hydrocortisone, 10 μg/mL insulin, and 100 ng/mL cholera toxin. E0771 (RRID: CVCL_GR23) cell line was maintained in DMEM medium (Gibco, 11995-065) with 10% fetal bovine serum (FBS). 4T1 (RRID: CVCL_0125), THP1 (RRID: CVCL_0006) and Jurkat (RRID: CVCL_0065) cell lines were maintained in RPMI medium with 10% FBS. All cells were cultured at 37° C. with 5% CO₂. Lentivirus was made by transfecting HEK293T cells with packaging and envelope plasmids and shRNA plasmids (see Table 1). Media containing virus was collected 48, filtered and virus particles pelleted with PEG—it (System Biosciences, #LV810Δ-1) according to manufacturer's instructions. Cells were infected with lentivirus then selected with 5 mg/ml puromycin (Thermofisher, #Δ11138-03).

TABLE 1
Plasmids used to generate the lentivirus.

| Manufacturer | Plasmid | Plasmid No. |
|---|---|---|
| Addgene | pLKO.1 puro shTEAD1/3/4-1 | 193672 |
| Addgene | pLKO.1 puro shTEAD1/3/4-2 | 193673 |
| Addgene | pLKO.1 puro shGFP | 30323 |
| Addgene | pVSV-G | 8454 |
| System Biosciences | pPACKF1 FIV | LV100A-1 |

RNA Extraction and qPCR

Total RNA was extracted from cell lines using a RNeasy Mini Kit (Qiagen, #74104) according to the manufacturer's instructions. cDNA from total RNA was made with the iScript cDNA synthesis kit according to the manufacturer's protocol (Biorad, #170-8891) and qPCR was performed in an iCycler iQ (BioRad) using the iQ SYBR Green Supermix (BioRad, #170-8882). The primers used are indicated in Table 2.

TABLE 2
Primers used for qPCR.

| Primer | Sequence |
|---|---|
| hCD80/FWD | AAACTCGCATCTACTGGCAAA (SEQ ID NO: 1) |
| hCD80/REV | GGTTCTTGTACTCGGGCCATA (SEQ ID NO: 2) |
| hIL-12A/FWD | CCAGAAGGCCAGACAAACTC (SEQ ID NO: 3) |
| hIL-12A/REV | GCCAGGCAACTCCCATTAG (SEQ ID NO: 4) |
| hS100A8/FWD | GGGATGACCTGAAGAAATTGCTA |
| | (SEQ ID NO: 5) |
| hS100A8/REV | TGTTGATATCCAACTCTTTGAACCA |
| | (SEQ ID NO: 6) |
| hS100A9/FWD | GTGCGAAAAGATCTGCAAAATTT |
| | (SEQ ID NO: 7) |
| hS100A9/REV | GGTCCTCCATGATGTGTTCTATGA |
| | (SEQ ID NO: 8) |
| hCTGF/FWD | CAGCATGGACGTTCGTCTG (SEQ ID NO: 9) |
| hCTGF/REV | AACCACGGTTTGGTCCTTGG (SEQ ID NO: 10) |
| hActin/FWD | CCTGGCACCCAGCACAAT (SEQ ID NO: 11) |
| hActin/REV | GCCGATCCACACGGAGTACT (SEQ ID NO: 12) |
| mActin/FWD | GGCGCTTTTGACTCAGGATTTAA |
| | (SEQ ID NO: 13) |
| mActin/REV | CCTCAGCCACATTTGTAGAACTTT |
| | (SEQ ID NO: 14) |

### 3D Sphere Co-Culture Assay

One thousand 4T1 cells were aggregated in 81-well agarose molds (Microtissues, #Z764019). Immune cells and treatments were added to the media according to the experiment's description. At the endpoint, spheres were fixed in 5% formalin and embedded in histogel (Fisher Scientific, #22-110-678) for sectioning (Lin et al., 2020). FFPE immunohistochemistry was done as previously described in this methods section with primary antibodies for Caspase 3(1/80, BIOCARE, CP229Δ). Images were captured using the Olympus BX40 microscope, and DAB signal per sphere was quantified in imageJ and normalized to the number of nuclei.

In the experiments where spheres were embedded in 20% Matrigel (Corning, #354230) and 80% collagen I mix (Thermofisher, #154453), agarose molds were removed before immune cells and treatments were added in the media and onto the matrix embedded spheres according to the experiment's description. THP1 cells were labeled with 1 ul of the CMFDA green tracer (Thermofisher, C7025) for 30 min, then washed with RPMI media.

### Statistics

Analyses were performed either using the R platform for statistical computing (version 3.6.1) and the indicated library packages implemented in Bioconductor (RRID:SCR_006442) or Prism 7 (Graphpad Inc, RRID:SCR_006442). Student t-tests and Fisher exact tests were used for comparisons as indicated in figure legends, with p<0.05 as the threshold for statistical significance in all tests.

### REFERENCES

Bum-Erdene K, et al., Small-molecule covalent modification of conserved cysteine leads to allosteric inhibition of the TEAD-YAP protein-protein interaction, Cell Chem Biol, 2019, 26(3): 378-389.
Caprica R, et al., Pulmonary nodules in patients with nonpulmonary cancer: not always metastases, J Glob Oncol, 2016, 2(3): 138-144.
Chan P, et al., Autopalmitoylation of TEAD proteins regulates transcriptional output of the Hippo pathway, Nat Chem Biol, 2016, 12(4): 282-289.
Chen B, et al., Profiling tumor infiltrating immune cells with CIBERSORT, Methods Mol Biol, 2018, 1711: 243-259.
Chen P-Y, et al., OPN-9840, a non-covalent potent pan-TEAD inhibitor, exhibits single agent efficacy in preclinical malignant mesothelioma models [abstract], In: Proceedings of the American Association for Cancer Research Annual Meeting 2024, Part 1 (Regular Abstracts), Cancer Res, 2024, 84(6_Suppl): abstract 7264.
Chen Y, et al., Inflammatory macrophages switch to CCL17-expressing phenotype and promote peritoneal fibrosis, J Pathol, 2020, 250(1): 55-66.
Chen Z, et al., Transcriptional enhancer factor-1 disruption by a retroviral gene trap leads to heart-defects and embryonic lethality in mice, Gene Dev, 1994, 8(19): 2293-2301.
Contento R L, et al., CXCR4-CCR5: a couple modulating T cell functions, Proc Natl Acad Sci, 2008, 105(29): 10101-10106.
Du X, et al., Mst1/Mst2 regulate development and function of regulatory t cells through modulation of Foxo1/Foxo3 stability in autoimmune disease, J Immunol, 2014, 192(4): 1525-1535.
Geng J, et al., The transcriptional coactivator TAZ regulates reciprocal differentiation of TH17 cells and Treg cells, Nat Immunol, 2017, 18(7): 800-812.
Guerriero J L, Chapter three macrophages their untold story in T cell activation and function, Int Rev Cell Mol Biol, 2019, 342: 73-93.
Holden J K, et al., Small molecule dysregulation of TEAD lipidation induces a dominant-negative inhibition of hippo pathway signaling, Cell Rep, 2020, 31(12): 107809.
Hu L, et al., Discovery of a new class of reversible TEA domain transcription factor inhibitors with a novel binding mode, eLife, 2022, 11: e80210.
Jacquemin P, et al., A novel family of developmentally regulated mammalian transcription factors containing the TEA/ATTS DNA binding domain, J Biol Chem, 1996, 271(36): 21775-21785.
Johnstone C N, et al., Functional and molecular characterisation of E0771.LMB tumours, a new C57BL/6-mouse-derived model of spontaneously metastatic mammary cancer, Dis Model Mech, 2015, 8(3): 237-251.
Kaczanowska S, et al., Genetically engineered myeloid cells rebalance the core immune suppression program in metastasis, Cell, 2021, 184(8): 2033-2052.e21.
Kaneda A, et al., The novel potent TEAD inhibitor, K-975, inhibits YAP1/TAZ-TEAD protein-protein interactions and exerts an anti-tumor effect on malignant pleural mesothelioma, Am J Cancer Res, 2020, 10(12): 4399-4415.
Kaneko K J, et al., Transcription factor TEAD2 is involved in neural tube closure, Genesis, 2007, 45(9): 577-587.
Kesireddy M, et al., Overall survival and prognostic factors in metastatic triple-negative breast cancer: a national cancer database analysis, Cancers, 2024, 16(10): 1791.
Kurppa K J, et al., Treatment-induced tumor dormancy through YAP-mediated transcriptional reprogramming of the apoptotic pathway, Cancer Cell, 2020, 37(1): 104-122.
Lebid A, et al., YAP attenuates CD8 T cell-mediated anti-tumor response, Front Immunol, 2020, 11: 580.
Li C H, et al., Current treatment landscape for patients with locally recurrent inoperable or metastatic triple-negative breast cancer: a systematic literature review, Breast Cancer Res, 2019, 21(1): 143.
Li Q, et al., Lats1/2 Sustain intestinal stem cells and Wnt activation through TEAD-dependent and independent transcription, Cell Stem Cell, 2020, 26(5): 675-692.
Lin S, et al., Targeting parvalbumin promotes M2 macrophage polarization and energy expenditure in mice, Nat Commun, 2022, 13(1): 3301.
Lin Y-N, et al., Monitoring cancer cell invasion and T-cell cytotoxicity in 3D culture, J Vis Exp, 2020, (160): 10.3791/61392.
Linsley P S & Ledbetter J A, The role of the CD28 receptor during t cell responses to antigen, Annu Rev Immunol, 1993, 11: 191-212.
Lu T, et al., Discovery of a subtype-selective, covalent inhibitor against palmitoylation pocket of TEAD3, Acta Pharm Sin B, 2021, 11(10): 3206-3219.
Lu W, et al., Discovery and biological evaluation of vinylsulfonamide derivatives as highly potent, covalent TEAD autopalmitoylation inhibitors, Eur J Med Chem, 2019, 184: 111767.
McGinnis C S, et al., The temporal progression of lung immune remodeling during breast cancer metastasis, Cancer Cell, 2024, 42(6): 1018-1031.e6.
Miller J E, et al., IL-17A modulates peritoneal macrophage recruitment and M2 polarization in endometriosis, Front Immunol, 2020, 11: 108.
Ni X, et al., YAP is essential for Treg mediated suppression of anti-tumor immunity, Cancer Discov, 2018, 8(8): 1026-1043.
Noland C L, et al., Palmitoylation of TEAD transcription factors is required for their stability and function in Hippo pathway signaling, Structure, 2016, 24(1): 179-186.
Noy R & Pollard J W, Tumor-associated macrophages: from mechanisms to therapy, Immunity, 2014, 41(1): 49-61.
O'Reilly D, et al., Overview of recent advances in metastatic triple negative breast cancer, World J Clin Oncol, 2021, 12(3): 164-182.
Oft M, IL-10: master switch from tumor-promoting inflammation to antitumor immunity, Cancer Immunol Res, 2014, 2(3): 194-199.
Oishi S, et al., M2 polarization of murine peritoneal macrophages induces regulatory cytokine production and suppresses T-cell proliferation, Immunology, 2016, 149(3): 320-328.
Paine A, et al., IL-2 upregulates CD86 expression on human CD4+ and CD8+ T cells, J Immunol, 2012, 188(4): 1620-1629.
PCT Publication No. WO 2019/040380 Δ1, published 28 Feb. 2019.
PCT Publication No. WO 2020/097389 Δ1, published 14 May 2020.
Peters P J, et al., Cytotoxic T lymphocyte granules are secretory lysosomes, containing both perforin and granzymes, J Exp Med, 1991, 173(5): 1099-1109.
Pobbati A V, et al., Targeting the central pocket in human transcription factor TEAD as a potential cancer therapeutic strategy, Structure, 2015, 23(11): 2076-2086.
Pulaski BA & Ostrand-Rosenberg S, Mouse 4T1 breast tumor model, Curr Protoc Immunol, 2000, 39: 20.2.1-20.2.16.
Qian B-Z, et al., CCL2 recruits inflammatory monocytes to facilitate breast-tumour metastasis, Nature, 2011, 475(7355): 222-225.
Ramos M I P, et al., Cancer immunotherapy by NC410, a LAIR-2 Fc protein blocking human LAIR-collagen interaction, eLife, 2021, 10: e62927.
Rashid OM & Takabe K, The evolution of the role of surgery in the management of breast cancer lung metastasis, J Thorac Dis, 2012, 4(4): 420-424.
Ries C H, et al., Targeting tumor-associated macrophages with anti-CSF-1R antibody reveals a strategy for cancer therapy, Cancer Cell, 2014, 25(6): 846-859.
Schneider U, et al., Characterization of EBV-genome negative “null” and “T” cell lines derived from children with acute lymphoblastic leukemia and leukemic transformed non-Hodgkin lymphoma, Int J Cancer, 1977, 19(5): 621-626.
Shao Y, et al., CXCL8 induces M2 macrophage polarization and inhibits CD8+ T cell infiltration to generate an immunosuppressive microenvironment in colorectal cancer, FASEB J, 2023, 37(10): e23173.
Sharif G M, et al., An AIB1 isoform alters enhancer access and enables progression of early-stage triple-negative breast cancer, Cancer Res, 2021, 81(16): 4230-4241.
Shrivastava R, et al., M2 polarization of macrophages by Oncostatin M in hypoxic tumor microenvironment is mediated by mTORC2 and promotes tumor growth and metastasis, Cytokine, 2019, 118: 130-143.
Sica A, et al., Cancer related inflammation: the macrophage connection, Cancer Lett, 2008, 267(2): 204-215.
Stella G M, et al., Lung-seeking metastases, Cancers (Basel), 2019, 11(7): 1010.
Szabo S J, et al., A novel transcription factor, T-bet, directs Th1 lineage commitment, Cell, 2000, 100(6): 655-669.
Tang T T, et al., Small Molecule Inhibitors of TEAD auto-palmitoylation selectively inhibit proliferation and tumor growth of NF2-deficient mesothelioma, Mol Cancer Ther, 2021, 20(6): 986-998.
Thelen B, et al., Eomes is sufficient to regulate IL-10 expression and cytotoxic effector molecules in murine CD4+ T cells, Front Immunol, 2023, 14: 1058267.
Tolcher A W, et al., A phase 1, first-in-human study of IK-930, an oral TEAD inhibitor targeting the Hippo pathway in subjects with advanced solid tumors, J Clin Oncol, 2022, 40(16_suppl): abstract TPS3168.
Tsuchiya S, et al., Establishment and characterization of a human acute monocytic leukemia cell line (THP-1), Int J Cancer, 1980, 26(2): 171-176.
Ueda Y, et al., Mst1 regulates integrin-dependent thymocyte trafficking and antigen recognition in the thymus, Nat Commun, 2012, 3: 1098.
Wang J, et al., Diagnostic and prognostic nomograms for lung metastasis in triple-negative breast cancer, Comput Math Methods Med, 2022, 2022: 1750834.
Xiao W, et al., Risk factors and survival outcomes in patients with breast cancer and lung metastasis: a population-based study, Cancer Med, 2018, 7: 922-930.
Xue D, et al., A tumor-specific pro-IL-12 activates preexisting cytotoxic T cells to control established tumors, Sci Immunol, 2022, 7(67):eabi6899.
Yagi R, et al., Transcription factor TEAD4 specifies the trophectoderm lineage at the beginning of mammalian development, Development, 2007, 134(21): 3827-3836.
Yin L, et al., Triple-negative breast cancer molecular subtyping and treatment progress, Breast Can Res, 2020, 22(1): 61.
Zhao X, et al., Clinical characteristics and prognoses of patients treated surgically for metastatic lung tumors, Oncotarget, 2017, 8(28): 46491-46497.

1-2. (canceled)

3. A method of inhibiting growth of a secondary tumor in a lung of a subject in need thereof, the method comprising administering a pharmaceutical composition comprising an effective amount of an inhibitor of transcriptional enhanced associate domain (TEAD) to the subject.

4-6. (canceled)

7. The method of claim 3, wherein the subject comprises a primary tumor in a tissue selected from breast, bone, esophagus, colon, rectum, kidney, cervix, prostate, larynx, liver, pancreas, brain, lung, and skin.

8. The method of claim 7, wherein the subject comprises a primary tumor in breast tissue.

9. The method of claim 8, wherein the subject has triple negative breast cancer.

10-17. (canceled)

18. A method of reducing lung metastases in a subject in need thereof, the method comprising administering a pharmaceutical composition comprising an effective amount of an inhibitor of transcriptional enhanced associate domain (TEAD) to the subject.

19. (canceled)

20. The method of claim 18, wherein the subject has metastatic cancer of the lung.

21. The method of claim 20, wherein the subject has primary cancer selected from breast, bone, esophageal, colon, rectal, kidney, cervical, prostate, larynx, liver, pancreatic, brain, lung, and skin cancer.

22. The method of claim 21, wherein the primary cancer is breast cancer.

23. The method of claim 22,
wherein the breast cancer is triple negative breast cancer.

24-25. (canceled)

26. A method of inducing polarization of one or more M2 macrophages to M1 macrophages in the lung of a subject with lung cancer, the method comprising administering a pharmaceutical composition comprising an effective amount of an inhibitor of transcriptional enhanced associate domain (TEAD) to the subject.

27-29. (canceled)

30. The method of claim 26, wherein the lung cancer is a primary cancer.

31. (canceled)

32. The method of claim 31, wherein the subject has primary cancer selected from breast, bone, esophageal, colon, rectal, kidney, cervical, prostate, larynx, liver, pancreatic, brain, lung, and skin cancer.

33. The method of claim 32, wherein the primary cancer is breast cancer.

34. The method of claim 33, wherein the breast cancer is triple negative breast cancer.

35. The method of claim 3, wherein the pharmaceutical composition is administered systemically.

36. The method of claim 35, wherein the pharmaceutical composition is administered via a route selected from intravenously, orally, intraperitoneally, intramuscularly, intradermally, intrathecally, subcutaneously, and nasally.

37. The method claim 18, wherein the pharmaceutical composition is administered systemically.

38. The method of claim 37, wherein the pharmaceutical composition is administered via a route selected from intravenously, orally, intraperitoneally, intramuscularly, intradermally, intrathecally, subcutaneously, and nasally.

39. The method claim 26, wherein the pharmaceutical composition is administered systemically.

40. The method of claim 39, wherein the pharmaceutical composition is administered via a route selected from intravenously, orally, intraperitoneally, intramuscularly, intradermally, intrathecally, subcutaneously, and nasally.


SEQUENCE LISTING (14 sequences)

SEQ ID NO: 1 - DNA, 21 bp, synthetic construct
  source 1..21: mol_type = other DNA
aaactcgcatctactggcaaa

SEQ ID NO: 2 - DNA, 21 bp, synthetic construct
  source 1..21: mol_type = other DNA
ggttcttgtactcgggccata

SEQ ID NO: 3 - DNA, 20 bp, synthetic construct
  source 1..20: mol_type = other DNA
ccagaaggccagacaaactc

SEQ ID NO: 4 - DNA, 19 bp, synthetic construct
  source 1..19: mol_type = other DNA
gccaggcaactcccattag

SEQ ID NO: 5 - DNA, 23 bp, synthetic construct
  source 1..23: mol_type = other DNA
gggatgacctgaagaaattgcta

SEQ ID NO: 6 - DNA, 25 bp, synthetic construct
  source 1..25: mol_type = other DNA
tgttgatatccaactctttgaacca

SEQ ID NO: 7 - DNA, 23 bp, synthetic construct
  source 1..23: mol_type = other DNA
gtgcgaaaagatctgcaaaattt

SEQ ID NO: 8 - DNA, 24 bp, synthetic construct
  source 1..24: mol_type = other DNA
ggtcctccatgatgtgttctatga

SEQ ID NO: 9 - DNA, 19 bp, synthetic construct
  source 1..19: mol_type = other DNA
cagcatggacgttcgtctg

SEQ ID NO: 10 - DNA, 20 bp, synthetic construct
  source 1..20: mol_type = other DNA
aaccacggtttggtccttgg

SEQ ID NO: 11 - DNA, 18 bp, synthetic construct
  source 1..18: mol_type = other DNA
cctggcacccagcacaat

SEQ ID NO: 12 - DNA, 20 bp, synthetic construct
  source 1..20: mol_type = other DNA
gccgatccacacggagtact

SEQ ID NO: 13 - DNA, 23 bp, synthetic construct
  source 1..23: mol_type = other DNA
ggcgcttttgactcaggatttaa

SEQ ID NO: 14 - DNA, 24 bp, synthetic construct
  source 1..24: mol_type = other DNA
cctcagccacatttgtagaacttt